(19) United States
(12) Patent Application Publication
(10) Pub. No.: US 2026/0234670 A1
(43) Pub. Date: Aug. 13, 2026

(54) ENRICHMENT OF GENETICALLY MODIFIED HEMATOPOIETIC STEM CELLS THROUGH MULTIPLEX BASE EDITING

(71) Applicants: INSTITUT NATIONAL DE LA SANTÉ ET DE LA RECHERCHE MÉDICALE, Paris (FR); UNIVERSITÉ PARIS CITÉ, Paris (FR); ASSISTANCE PUBLIQUE-HÔPITAUX DE PARIS (APHP), Paris (FR); FONDATION IMAGINE, Paris (FR)

(72) Inventors: Annarita MICCIO, Paris (FR); Panagiotis ANTONIOU, Mölndal (SE)

(21) Appl. No.: 19/150,580

(22) PCT Filed: Feb. 5, 2024
(86) PCT No.: PCT/EP2024/052752

§ 371 Date: Jul. 24, 2025

(30) Foreign Application Priority Data
Feb. 6, 2023 (EP) 23305158.0

(51) Int. Cl. C12N 15/90; A61K 35/28; A61K 47/68; C12N 9/22; C12N 9/78; C12N 15/11

(52) U.S. Cl. CPC C12N 15/907; A61K 35/28; A61K 47/6809; A61K 47/6849; C12N 9/226; C12N 9/78; C12N 15/11; C12Y 305/04001; C12Y 305/04002; C07K 2319/00; C12N 2310/20

(57) ABSTRACT

Here, the inventors developed a strategy to enrich for base edited HSPCs in order to achieve higher BE efficiencies by downregulating CD33 expression. Using the DSB-free nature of the BE system that allows multiplex editing without generating genomic rearrangements, the inventors indeed simultaneously targeted: (i) the HBG promoters, so as to insert HPFH and HPFH-like mutations that reactivate HbF, and (ii) the CD33 gene, so as to downregulate the expression of the CD33 surface marker. By selecting CD33 KO cells, the inventors enriched for populations edited at the HBG promoters and they eliminated unedited cells that normally outcompete edited cells during transplantation. This strategy will allow the ex vivo selection of corrected HSCs prior to transplantation. Furthermore, the inventors envision to use this strategy to enrich for in vivo edited cells vs unedited HSCs by administrating the toxin-conjugated anti-CD33 monoclonal antibody. The latter would target not only unedited HSCs derived from the drug product but also the endogenous HSCs, therefore potentially reducing the need of a full conditioning regimen and avoiding its side effects.

### FIELD OF THE INVENTION

The present invention is in the field of medicine, in particular gene editing and haematology.

### BACKGROUND OF THE INVENTION

SCD and β-thalassemia are genetic diseases caused by mutations in the β-globin locus. In SCD, a point mutation in the HBB gene leads to the formation of the sickle β^{S}-globin chain, which causes the polymerization of sickle hemoglobin (HbS), red blood cell (RBC) sickling, anemia, and organ damage^{1,2}. In β-thalassemia, the partial or total absence of β-globin chains (β⁺ and β⁰, respectively) leads to the precipitation of noncoupled α-globin chains, apoptosis of erythroid precursors, ineffective erythropoiesis, and anemia³⁻⁵. Gene therapy approaches based on the transplantation of autologous, genetically modified hematopoietic stem cells (HSCs) have been investigated as a treatment option for patients lacking a compatible donor for allogeneic HSC transplantation⁶.

The severity of both SCD and β-thalassemia is lessened by the hereditary persistence of fetal hemoglobin (HbF) in adulthood (HPFH) 7. This persistence is due to mutations located 200 to 115 nucleotides upstream of the transcription start sites of the identical HBG1 and HBG2 γ-globin promoters. HPFH mutations either generate de novo DNA motifs recognized by transcriptional activators (e.g., KLF1)⁸⁻¹⁰ or disrupt binding sites (BS) for transcriptional repressors (e.g., LRF and BCL11A)¹¹. CRISPR-Cas9 nuclease strategies have been used to disrupt the LRF and BCL11A repressor BS via the non-homologous end-joining (NHEJ)-mediated generation of insertions/deletions (InDels) that mimic HPFH mutations and reactivate HbF expression¹²⁻¹⁵.

HSCs are highly sensitive to DNA double-strand breaks (DSBs) 16-especially in case of multiple on-target events or concomitant on-target and off-target events. Even when highly specific single guide RNAs (sgRNAs) are used, the Cas9-sgRNA treatment of human HSPCs induces a DNA damage response (DDR) that can lead to apoptosis^{17,18}. CRISPR-Cas9 can cause p53-dependent cell toxicity and cell cycle arrest, resulting in the selection of cells with a dysfunctional p53 pathway¹⁹. Furthermore, the generation of several on-target DSBs, simultaneous on-target and off-target DSBs, or even a single on-target DSB can lead to genomic deletions, inversions or translocations, chromosome loss, and chromothripsis²⁰⁻²³. Hence, the development of novel, efficacious, safe treatment strategies for β-hemoglobinopathies based on precise base editing (rather than DSB-induced DNA repair) is highly desirable. Cytidine and adenine base editors (CBEs and ABEs) are composed of a Cas9 nickase and a deaminase, and introduce C-to-T and A-to-G point mutations²⁴, respectively. Importantly, base editors do not generate DNA DSBs or induce DDR and any consequent event. Hence, base editors allow the simultaneous editing of multiplex targets, an approach that would lead to genomic rearrangements in the case of the CRISPR/Cas9 nuclease system.

We recently reported adenine and cytidine ABE- and CBE-mediated introduction of HPFH and HPFH-like mutations in the −200 region of the HBG1/2 promoters with minimal off-target activity. This led to therapeutic HbF levels in human RBCs differentiated from base edited SCD or β-thalassemia hematopoietic stem/progenitor cells (HSPCs; containing mostly hematopoietic progenitors and less than 1% of HSCs) and rescued the pathological phenotype in vitro. However, even though xenotransplantation experiments (of human HSPCs in immunodeficient mice) showed BE in long-term HSCs, the efficiency was reduced compared to input HSPCs²⁵. BEs might have induced some toxicity in bona fide HSCs or might be less efficient in in this cell population compared to hematopoietic progenitors.

More in general, several limitations still hamper the broad application of genome editing approaches for hematopoietic disorders: (i) the efficiency in HSCs and (ii) in vivo competition of the infused unedited HSCs or the endogenous HSCs that are not completely eliminated with the myeloablation treatments that patients undergo to. Genetic modification of HSCs can potentially affect their properties. In particular, editing strategies are based on a 48- to 72-h in vitro treatment including the electroporation procedure, the introduction of the editing system complexes, the genetic modification of the target regions, and the exposure to cytokines. In this context non-edited HSCs might have a survival advantage and better stemness compared to edited cells.

Several parameters could be further optimized with regard to the base-editing efficiency in HSCs, and the fitness of the edited HSCs. Furthermore, enriching for ex vivo or in vivo edited versus non-edited HSCs could be exploited to maximize the engraftment of genetically modified cells. This could also allow the reduction of the conditioning regimen and its side effects. Such an approach could increase the success of therapeutic strategies for diseases with no or little selective advantage of corrected cells such as β-hemoglobinopathies^{26,27}.

CD33 is a surface marker of the myeloid lineage, which is also highly expressed in malignant blasts of acute myeloid leukemia (AML) patients²⁸⁻³⁰. Gemtuzumab, a toxin-conjugated anti-CD33 monoclonal antibody has been approved for treating AML. Of note, CD33 is also expressed on human HSCs with a high regenerative potential³¹ and one of the main side effects of Gemtuzumab is myelosuppression. Therefore, even though it lacks expression specificity, CD33 has been used as a target in patients with AML that are treated with Gemtuzumab, and in clinical trials (NCT03971799, NCT03927261) and experimental models of chimeric antigen receptor (CAR) T-cell immunotherapies^{32,33}. Interestingly, to avoid myelosuppression, researchers have combined transplantation of CD33 knock-out (KO) HSPCs with CAR T-cells efficiently targeting the from-now-on leukemia-specific CD33-expressing blasts. Importantly, CD33 KO HSPCs remained functional and were able to engraft and differentiate in animal models (mice and non-human primates)³². Furthermore, an ongoing clinical trial (NCT04849910) is based on the transplantation of CD33 KO HSPCs in AML patients with high risk of relapse, who require Gemtuzumab treatment post-transplantation, so as to reduce the toxic side effect of the antibody.

### SUMMARY OF THE INVENTION

The present invention is defined by the claims. In particular, the present invention relates to

### DETAILED DESCRIPTION OF THE INVENTION

Here, the inventors developed a strategy to enrich for base edited HSPCs in order to achieve higher BE efficiencies by downregulating CD33 expression. CD33 is a surface marker of the myeloid lineage, which is expressed on human HSCs with a high regenerative potential³¹. Importantly, CD33 KO HSPCs remained functional and were able to engraft and differentiate in animal models (mice and non-human primates) 32. Based on these findings, using the DSB-free nature of the BE system that allows multiplex editing without generating genomic rearrangements³², the inventors simultaneously targeted: (i) the HBG promoters, so as to insert HPFH and HPFH-like mutations that reactivate HbF, and (ii) the CD33 gene, so as to downregulate the expression of the CD33 surface marker. By selecting CD33 KO cells, the inventors enriched for populations edited at the HBG promoters and they eliminated unedited cells that normally outcompete edited cells during transplantation. This strategy will allow the ex vivo selection of corrected HSCs prior to transplantation. Furthermore, the inventors envision to use this strategy to enrich for in vivo edited cells vs unedited HSCs by administrating the toxin-conjugated anti-CD33 monoclonal antibody. The latter would target not only unedited HSCs derived from the drug product but also the endogenous HSCs, therefore potentially reducing the need of a full conditioning regimen and avoiding its side effects.

### Main Definitions

As used herein, the terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, pegylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

As used herein, the term “nucleic acid molecule” or “polynucleotide” refers to a DNA molecule (for example, but not limited to, a cDNA or genomic DNA). The nucleic acid molecule can be single-stranded or double-stranded.

As used herein, the term “exon” refers to a defined section of nucleic acid that encodes for a protein, or a nucleic acid sequence that is represented in the mature form of an RNA molecule after either portions of a pre-processed (or precursor) RNA have been removed by splicing. The mature RNA molecule can be a messenger RNA (mRNA) or a functional form of a non-coding RNA, such as rRNA or tRNA.

As used herein, the term “intron” refers to a nucleic acid region (within a gene) that is not translated into a protein. An intron is a non-coding section that is transcribed into a precursor mRNA (pre-mRNA), and subsequently removed by splicing during formation of the mature RNA.

As used herein, the term “splice site” refers to the short conserved sequence at the 5′ end (donor site) or 3′ end (acceptor site) of an intron to which a spliceosome binds and catalyzes the splicing of the intron from the pre-mRNA.

As used herein, the term “encode”, or “encoding” or “encoded” refers to a nucleic acid sequence that codes for a polypeptide sequence.

As used herein, the term “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell. Any method known in the art can be used to measure the expression of the gene (e. g. HPLC analysis of protein and RT-qPCR analysis of mRNA.) Typically, said methods are described in the EXAMPLE.

As used herein, the terms “decrease”, “reduced”, “reduction” “repress” are all used generally to mean a decrease by a statistically significant amount, for example, a decrease by at least 10%, or at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level. As used herein, the term “knock-down” as used herein refers to reduction in the expression of a gene or its gene product(s).

As used herein, the terms “increased”, “increase” or “enhance” or “activate” are all used to generally mean an increase by a statically significant amount, for example, an increase of at least 10%, or at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the terms “expressing (positive or +)” and “not expressing (negative or −)” are well known in the art and refer to the expression level of a phenotypic marker of interest (e.g. CD33), in that the expression level of the phenotypic marker corresponding to “+” is high or intermediate, also referred as “+/−”. The phenotypic marker corresponding to “−” is a null expression level of the phenotypic marker or also refers to less than 10% of a cell population expressing the said phenotypic marker.

As used herein, the term “antibody” herein is used to refer to a molecule having a useful antigen binding specificity. Those skilled in the art will readily appreciate that this term may also cover polypeptides which are fragments of or derivatives of antibodies yet which can show the same or a closely similar functionality. Such antibody fragments or derivatives are intended to be encompassed by the term antibody as used herein. By “antibody” or “antibody molecule”, it is intended herein not only whole immunoglobulin molecules but also fragments thereof, such as Fab, F(ab′)2, Fv and other fragments thereof. Similarly, the term antibody includes genetically engineered derivatives of antibodies such as single chain Fv molecules (scFv) and domain antibodies (dAbs). The term “monoclonal antibody” is used herein to encompass any isolated Ab's such as conventional monoclonal antibody hybridomas, but also to encompass isolated monospecific antibodies produced by any cell, such as for example a sample of identical human immunoglobulins expressed in a mammalian cell line. Suitable monoclonal antibodies which are reactive as described herein may be prepared by known techniques, for example those disclosed in “Monoclonal Antibodies; A manual of techniques”, H Zola (CRC Press, 1988) and in “Monoclonal Hybridoma Antibodies: Techniques and Application”, S G R Hurrell (CRC Press, 1982).

As used herein, the term “complementarity” refers to the ability of a nucleic acid to form hydrogen bond(s) with another nucleic acid sequence by either traditional Watson-Crick base-pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, the term “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part I, Second Chapter “Overview of principles of hybridization and the strategy of nucleic acid probe assay”, Elsevier, N.Y.

As used herein, the term “hybridization” or “hybridizing” refers to a process where completely or partially complementary nucleic acid strands come together under specified hybridization conditions to form a double-stranded structure or region in which the two constituent strands are joined by hydrogen bonds. Although hydrogen bonds typically form between adenine and thymine or uracil (A and T or U) or cytosine and guanine (C and G), other base pairs may form (e.g., Adams et al., The Biochemistry of the Nucleic Acids, 11th ed., 1992).

As used herein, the term “fusion polypeptide” or “fusion protein” means a protein created by joining two or more polypeptide sequences together. The fusion polypeptides encompassed in this invention include translation products of a chimeric gene construct that joins the nucleic acid sequences encoding a first polypeptide, e.g., an RNA-binding domain, with the nucleic acid sequence encoding a second polypeptide, e.g., an effector domain, to form a single open-reading frame. In other words, a “fusion polypeptide” or “fusion protein” is a recombinant protein of two or more proteins which are joined by a peptide bond or via several peptides. The fusion protein may also comprise a peptide linker between the two domains.

As used herein the term “wild type” is a term of the art understood by skilled persons and means the typical form of an organism, strain, gene or characteristic as it occurs in nature as distinguished from mutant or variant forms.

As used herein, the term “derived from” refers to a process whereby a first component (e.g., a first molecule), or information from that first component, is used to isolate, derive or make a different second component (e.g., a second molecule that is different from the first).

As used herein, the “percent identity” between the two sequences is a function of the number of identical positions shared by the sequences (i.e., % identity=number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm, as described below. The percent identity between two amino acid sequences can be determined using the Needleman and Wunsch algorithm (Needleman, Saul B. & Wunsch, Christian D. (1970). “A general method applicable to the search for similarities in the amino acid sequence of two proteins”. Journal of Molecular Biology. 48 (3): 443-53.). The percent identity between two nucleotide or amino acid sequences may also be determined using for example algorithms such as EMBOSS Needle (pair wise alignment; available at www.ebi.ac.uk). For example, EMBOSS Needle may be used with a BLOSUM62 matrix, a “gap open penalty” of 10, a “gap extend penalty” of 0.5, a false “end gap penalty”, an “end gap open penalty” of 10 and an “end gap extend penalty” of 0.5. In general, the “percent identity” is a function of the number of matching positions divided by the number of positions compared and multiplied by 100. For instance, if 6 out of 10 sequence positions are identical between the two compared sequences after alignment, then the identity is 60%. The % identity is typically determined over the whole length of the query sequence on which the analysis is performed. Two molecules having the same primary amino acid sequence or nucleic acid sequence are identical irrespective of any chemical and/or biological modification. According to the invention a first amino acid sequence having at least 90% of identity with a second amino acid sequence means that the first sequence has 90; 91; 92; 93; 94; 95; 96; 97; 98; 99 or 100% of identity with the second amino acid sequence.

As used herein, the term “linker” refers to any means, entity or moiety used to join two or more entities. A linker can be a covalent linker or a non-covalent linker. Examples of covalent linkers include covalent bonds or a linker moiety covalently attached to one or more of the proteins or domains to be linked. The linker can also be a non-covalent bond, e.g., an organometallic bond through a metal center such as platinum atom. For covalent linkages, various functionalities can be used, such as amide groups, including carbonic acid derivatives, ethers, esters, including organic and inorganic esters, amino, urethane, urea and the like. To provide for linking, the domains can be modified by oxidation, hydroxylation, substitution, reduction etc. to provide a site for coupling. Methods for conjugation are well known by persons skilled in the art and are encompassed for use in the present invention. Linker moieties include, but are not limited to, chemical linker moieties, or for example a peptide linker moiety (a linker sequence). It will be appreciated that modification which do not significantly decrease the function of the RNA-binding domain and effector domain are preferred.

As used herein, the “linked” as used herein refers to the attachment of two or more entities to form one entity. A conjugate encompasses both peptide-small molecule conjugates as well as peptide-protein/peptide conjugates.

As used herein, the term “editing”, “edit”, “edition”, or “edited” refers to a method of altering a nucleic acid sequence of a polynucleotide (e.g., a naturally-occurring wild type nucleic acid sequence or a naturally-occurring mutated nucleic acid sequence by introducing a change to a specific genomic target; the genomic target may include a chromosomal region, a coding polynucleotide (e.g., a gene), a promotor, a non-coding polynucleotide, or any nucleic acid sequence. The changes to a nucleic acid may include deletion, addition and other changes to the nucleic acid sequence in the genome.

As used herein, the term “base-editing enzyme” refers to fusion protein comprising a defective CRISPR/Cas nuclease linked to a deaminase polypeptide. The term is also known as “base-editor”. Two classes of base-editing enzymes—cytosine base-editing enzymes (CBEs) and adenine base-editing enzymes (ABEs)—can be used to generate single base pair edits without double stranded breaks. Typically, cytosine base-editing enzymes are created by fusing the defective CRISPR/Cas nuclease to a deaminase.

As used herein, the term “deaminase” refers to an enzyme that catalyses a deamination reaction. The term “deamination”, as used herein, refers to the removal of an amine group from one molecule. In some embodiments, the deaminase is a cytidine deaminase, catalysing the hydrolytic deamination of cytidine or deoxycytidine to uracil or deoxyuracil, respectively. In some embodiments, the deaminase is an adenosine deaminase, catalysing the hydrolytic deamination of adenosine to inosine, which is treated like guanosine by the cell, creating an A to G (or T to C) change.

As used herein, the term “nuclease” includes a protein (i.e. an enzyme) that induces a break in a nucleic acid sequence, e.g., a single or a double strand break in a double-stranded DNA sequence.

As used herein, the term “CRISPR/Cas nuclease” has its general meaning in the art and refers to segments of prokaryotic DNA containing clustered regularly interspaced short palindromic repeats (CRISPR) and associated nucleases encoded by Cas genes. In bacteria the CRISPR/Cas loci encode RNA-guided adaptive immune systems against mobile genetic elements (viruses, transposable elements and conjugative plasmids). Three types of CRISPR systems have been identified. CRISPR clusters contain spacers, the sequences complementary to antecedent mobile elements. CRISPR clusters are transcribed and processed into mature CRISPR (Clustered Regularly Interspaced Short Palindromic Repeats) RNA (crRNA). The CRISPR/Cas nucleases Cas9 and Cpf1 belong to the type II and type V CRISPR/Cas system and have strong endonuclease activity to cut target DNA. Cas9 is guided by a mature crRNA that contains about 20 nucleotides of unique target sequence (called spacer) and a trans-activating small RNA (tracrRNA) that also serves as a guide for ribonuclease III-aided processing of pre-crRNA. The crRNA: tracrRNA duplex directs Cas9 to target DNA via complementary base pairing between the spacer on the crRNA and the complementary sequence (called protospacer) on the target DNA. Cas9 recognizes a trinucleotide (NGG for S. pyogenes Cas9) protospacer adjacent motif (PAM) to specify the cut site (the 3^{rd} or the 4^{th} nucleotide upstream from PAM).

As used herein, the term “Cas9” or “Cas9 nuclease” refers to an RNA-guided nuclease comprising a Cas9 protein, or a fragment thereof (e.g., a protein comprising an active or inactive DNA cleavage domain of Cas9, and/or the gRNA binding domain of Cas9). A Cas9 nuclease is also referred to sometimes as a casn1 nuclease or a CRISPR (clustered regularly interspaced short palindromic repeat)-associated nuclease. CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species. See, e.g., Jinek M., Chylinski K., Fonfara I., Hauer M., Doudna J. A., Charpentier E. Science 337:816-821 (2012), the entire contents of which is hereby incorporated by reference. Cas9 recognizes a short motif in the CRISPR repeat sequences (the PAM or protospacer adjacent motif) to help distinguish self versus non-self. Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., Mclaughlin R. E., Proc. Natl. Acad. Sci. U.S.A. 98:4658-4663 (2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607 (2011); and “A programmable dual-RNA-guided DNA endonuclease in adaptive bacterial immunity.” Jinek M., Chylinski K., Fonfara I., Hauer M., Doudna J. A., Charpentier E. Science 337:816-821 (2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, the term “Cas9” refers to Cas9 from: Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquisI (NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); or Neisseria. meningitidis (NCBI Ref: YP_002342100.1).

As used herein, the term “defective CRISPR/Cas nuclease” refers to a CRISPR/Cas nuclease having lost at least one nuclease domain.

As used herein, the term “nickase” has its general meaning in the art and refers to an endonuclease which cleaves only a single strand of a DNA duplex. Accordingly, the term “Cas9 nickase” refers to a nickase derived from a Cas9 protein, typically by inactivating one nuclease domain of Cas9 protein.

As used herein, the term “guide RNA molecule” generally refers to an RNA molecule (or a group of RNA molecules collectively) that can bind to a Cas9 protein and target the Cas9 protein to a specific location within a target DNA. A guide RNA can comprise two segments: a DNA-targeting guide segment and a protein-binding segment. The DNA-targeting segment comprises a nucleotide sequence that is complementary to (or at least can hybridize to under stringent conditions) a target sequence. The protein-binding segment interacts with a CRISPR protein, such as a Cas9 or Cas9 related polypeptide. These two segments can be located in the same RNA molecule or in two or more separate RNA molecules. When the two segments are in separate RNA molecules, the molecule comprising the DNA-targeting guide segment is sometimes referred to as the CRISPR RNA (crRNA), while the molecule comprising the protein-binding segment is referred to as the trans-activating RNA (tracrRNA).

As used herein, the term “target sequence” or “target” refers to a nucleic acid containing a target nucleic acid sequence. A target nucleic acid may be single-stranded or double-stranded, and often is double-stranded DNA. A “target nucleic acid sequence,” “target sequence” or “target region” as used herein, means a specific sequence or the complement thereof that one wishes to bind to using the CRISPR system as disclosed herein. As used herein, the term “target nucleic acid strand” refers to a strand of a target nucleic acid that is subject to base-pairing with a guide RNA as disclosed herein. That is, the strand of a target nucleic acid that hybridizes with the crRNA and guide sequence is referred to as the “target nucleic acid strand.” The other strand of the target nucleic acid, which is not complementary to the guide sequence, is referred to as the “non-complementary strand.” In the case of double-stranded target nucleic acid (e.g., DNA), each strand can be a “target nucleic acid strand” to design crRNA and guide RNAs and used to practice the method of this invention as long as there is a suitable PAM site.

As used herein, the term “ribonucleoprotein complex,” or “ribonucleoprotein particle” refers to a complex or particle including a nucleoprotein and a ribonucleic acid. A “nucleoprotein” as provided herein refers to a protein capable of binding a nucleic acid (e.g., RNA, DNA). Where the nucleoprotein binds a ribonucleic acid it is referred to as “ribonucleoprotein.” The interaction between the ribonucleoprotein and the ribonucleic acid may be direct, e.g., by covalent bond, or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like).

As used herein, the term “CD33” has its general meaning in the art and refers to the transmembrane receptor encoded by the CD33 gene. The term is also known as Siglec-3 (sialic acid binding Ig-like lectin 3), SIGLEC3, SIGLEC-3, gp67, or p67). An exemplary amino acid sequence for CD33 is shown as SEQ ID NO:1.

>sp|P20138|CD33 HUMAN Myeloid cell surface antigen CD33
SEQ ID NO: 1
OS = Homo sapiens OX = 9606 GN = CD33 PE = 1 SV = 2
MPLLLLLPLLWAGALAMDPNFWLQVQESVTVQEGLCVLVPCTFFHPIPYYDKNSPVHGYW
FREGAIISRDSPVATNKLDQEVQEETQGRFRLLGDPSRNNCSLSIVDARRRDNGSYFFRM
ERGSTKYSYKSPQLSVHVTDLTHRPKILIPGTLEPGHSKNLTCSVSWACEQGTPPIFSWL
SAAPTSLGPRTTHSSVLIITPRPQDHGTNLTCQVKFAGAGVTTERTIQLNVTYVPQNPTT
GIFPGDGSGKQETRAGVVHGAIGGAGVTALLALCLCLIFFIVKTHRRKAARTAVGRNDTH
PTTGSASPKHQKKSKLHGPTETSSCSGAAPTVEMDEELHYASLNFHGMNPSKDTSTEYSE
VRTQ

As used herein, the term “mutation” has its general meaning in the art and refers to a substitution, deletion or insertion. The term “substitution” means that a specific amino acid residue at a specific position is removed and another amino acid residue is inserted into the same position. The term “deletion” means that a specific amino acid residue is removed. The term “insertion” means that one or more amino acid residues are inserted before or after a specific amino acid residue. As used herein, the term “point mutation” refers to a substitution that replaces one of the nucleotides in a target polynucleotide.

As used herein, the term “mutagenesis” refers to the introduction of mutations into a polynucleotide sequence.

As used herein, the term “variant” refers to a first composition (e.g., a first molecule), that is related to a second composition (e.g., a second molecule, also termed a “parent” molecule). The variant molecule can be derived from, isolated from, based on or homologous to the parent molecule. A variant molecule can have entire sequence identity with the original parent molecule, or alternatively, can have less than 100% sequence identity with the parent molecule. For example, a variant of a sequence can be a second sequence that is at least 50; 51; 52; 53; 54; 55; 56; 57; 58; 59; 60; 61; 62; 63; 64; 65; 66; 67; 68; 69; 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; 100% identical in sequence compare to the original sequence.

As used herein, the term “hematopoietic stem cell” or “HSC” refers to blood cells that have the capacity to self-renew and to differentiate into precursors of blood cells. These precursor cells are immature blood cells that cannot self-renew and must differentiate into mature blood cells. Hematopoietic stem progenitor cells display a number of phenotypes, such as Lin-CD34+CD38−CD90+CD45RA−, Lin-CD34+CD38−CD90−CD45RA−, Lin-CD34+CD38+IL-3aloCD45RA−, and Lin-CD34+CD38+CD10+ (Daley et al., Focus 18:62-67, 1996; Pimentel, E., Ed., Handbook of Growth Factors Vol. III: Hematopoietic Growth Factors and Cytokines, pp. 1-2, CRC Press, Boca Raton, Fla., 1994). Within the bone marrow microenvironment, the stem cells self-renew and maintain continuous production of hematopoietic stem cells that give rise to all mature blood cells throughout life. In some embodiments, the hematopoietic progenitor cells or hematopoietic stem cells are isolated form peripheral blood cells.

As used herein, the term “isolated cell” refers to a cell that has been removed from an organism in which it was originally found, or a descendant of such a cell. Optionally the eukaryotic cell has been cultured in vitro, e.g., in the presence of other cells. Optionally the eukaryotic cell is later introduced into a second organism or reintroduced into the organism from which it (or the cell from which it is descended) was isolated. As used herein, the term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched.

As used herein, the expression “substantially pure population of cells” means a population of cells that contains at least 90, 91, 92, 03, 94, 95, 96, 97, 98, or 99% of the desired cell type.

As used herein, the term “enriching” includes any isolation or sorting process that increases the relative abundance of a desired cell type, or cell types, in a population of cells.

As used herein, the term “alpha globin” or “α-globin” has its general meaning in the art and refers to protein that is encoded in human by the HBA1 and HBA2 genes. The human alpha globin gene cluster located on chromosome 16 spans about 30 kb and includes seven loci: 5′-zeta-pseudozeta-mu-pseudoalpha-1-alpha-2-alpha-1-theta-3′. The alpha-2 (HBA2) and alpha-1 (HBA1) coding sequences are identical. These genes differ slightly over the 5′ untranslated regions and the introns, but they differ significantly over the 3′ untranslated regions. The ENSEMBL IDs (i.e. the gene identifier number from the Ensembl Genome Browser database) for HBA1 and HBA2 are ENSG00000206172 and ENSG00000188536 respectively.

As used herein, the term “beta globin” or “β-globin”” has its general meaning in the art and refers to a globin protein, which along with alpha globin (HBA), makes up the most common form of haemoglobin (Hb) in adult humans. Normal adult human Hb is a heterotetramer consisting of two alpha chains and two beta chains. HBB is encoded by the HBB gene on human chromosome 11. It is 146 amino acids long and has a molecular weight of 15,867 Da.

As used herein, the term “gamma globin” or “γ-globin” has its general meaning in the art and refers to protein that is encoded in human by the HBG1 and HBG2 genes. The HBG1 and HBG2 genes are normally expressed in the fetal liver, spleen and bone marrow. Two γ-globin chains together with two α-globin chains constitute fetal hemoglobin (HbF) which is normally replaced by adult hemoglobin (HbA) in the year following birth. The ENSEMBL IDs (i.e. the gene identifier number from the Ensembl Genome Browser database) for HBG1 and HBG2 are ENSG00000213934 and ENSG00000196565 respectively.

As used herein, the term “β-hemoglobinopathy” has its general meaning in the art and refers to any defect in the structure or function of any hemoglobin of an individual, and includes defects in the primary, secondary, tertiary or quaternary structure of hemoglobin caused by any mutation, such as deletion mutations or substitution mutations in the coding regions of the HBB gene, or mutations in, or deletions of, the promoters or enhancers of such gene that cause a reduction in the amount of hemoglobin produced as compared to a normal or standard condition.

As used herein, the term “sickle cell disease” has its general meaning in the art and refers to a group of autosomal recessive genetic blood disorders, which results from mutations in a globin gene and which is characterized by red blood cells that assume an abnormal, rigid, sickle shape. They are defined by the presence of BS-globin gene coding for a β-globin chain variant in which glutamic acid is substituted by valine at amino acid position 6 of the peptide: incorporation of the BS-globin in the Hb tetramers (HbS, sickle Hb) leads to Hb polymerization and to a clinical phenotype. The term includes sickle cell anemia (HbSS), sickle-hemoglobin C disease (HbSC), sickle beta-plus-thalassaemia (HbS/β+), or sickle beta-zerothalassaemia (HbS/β0).

As used herein, the term “β-thalassemia” refers to a hemoglobinopathy that results from an altered ratio of α-globin to β-like globin polypeptide chains resulting in the underproduction of normal hemoglobin tetrameric proteins and the precipitation of free, unpaired α-globin chains.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “therapeutically effective amount” is meant a sufficient amount of population of cells to treat the disease at a reasonable benefit/risk ratio applicable to any medical treatment. It will be understood that the total usage the gene editing platform will be decided by the attending physician within the scope of sound medical judgment. The specific therapeutically effective dose level for any particular patient will depend upon a variety of factors including the age, body weight, general health, sex and diet of the patient, the time of administration, route of administration, the duration of the treatment, drugs used in combination or coincidental with the population of cells, and like factors well known in the medical arts. In some embodiments, the cells are formulated by first harvesting them from their culture medium, and then washing and concentrating the cells in a medium and container system suitable for administration (a “pharmaceutically acceptable” carrier) in a treatment-effective amount. Suitable infusion medium can be any isotonic medium formulation, typically normal saline, Normosol R (Abbott) or Plasma-Lyte A (Baxter), but also 5% dextrose in water or Ringer's lactate can be utilized. The infusion medium can be supplemented with human serum albumin. A treatment-effective amount of cells in the composition is dependent on the relative representation of the cells with the desired specificity, on the age and weight of the recipient, and on the severity of the targeted condition. This number of cells can be as low as approximately 10³/kg, preferably 5×10³/kg; and as high as 10⁷/kg, preferably 10⁸/kg. The number of cells will depend upon the ultimate use for which the composition is intended, as will the type of cells included therein. Typically, the minimal dose is 2 millions of cells per kg. Usually 2 to 20 millions of cells are injected in the subject. The desired purity can be achieved by introducing a sorting step. For uses provided herein, the cells are generally in a volume of a liter or less, can be 500 ml or less, even 250 ml or 100 ml or less. The clinically relevant number of cells can be apportioned into multiple infusions that cumulatively equal or exceed the desired total amount of cells.

### Methods

The first object of the present invention relates to a method for editing an eukaryotic cell comprising contacting said eukaryotic cell with a gene editing platform that comprises (a) at least one base-editing enzyme, and (b) a plurality of guide RNA molecules designed for guiding the base-editing enzyme(s) to a plurality of target sequences wherein at least one guide RNA molecule is designed for guiding the base-editing enzyme(s) to a target sequence in the gene encoding for CD33 so as to knock-down the expression of CD33.

### Eukaryotic Cell:

In some embodiments, the eukaryotic cell is selected from the group consisting of hematopoietic progenitor cells, hematopoietic stem cells (HSCs), pluripotent cells (i.e. embryonic stem cells (ES) and induced pluripotent stem cells (iPS)). More preferably the eukaryotic cell is a hematopoietic stem cell.

In some embodiments, the hematopoietic progenitor cells or hematopoietic stem cells are isolated form peripheral blood cells. As used herein, the term “peripheral blood cells” refer to the cellular components of blood, including red blood cells, white blood cells, and platelets, which are found within the circulating pool of blood.

In some embodiments, the eukaryotic cell is a bone marrow derived stem cell. As used herein the term “bone marrow-derived stem cells” refers to stem cells found in the bone marrow. Stem cells may reside in the bone marrow, either as an adherent stromal cell type that possess pluripotent capabilities, or as cells that express CD34 or CD45 cell-surface protein, which identifies hematopoietic stem cells able to differentiate into blood cells.

Typically, the eukaryotic cell results from a stem cell mobilization.

As used herein, the term “mobilization” or “stem cell mobilization” refers to a process involving the recruitment of stem cells from their tissue or organ of residence to peripheral blood following treatment with a mobilization agent. This process mimics the enhancement of the physiological release of stem cells from tissues or organs in response to stress signals during injury and inflammation. The mechanism of the mobilization process depends on the type of mobilization agent administered. Some mobilization agents act as agonists or antagonists that prevent the attachment of stem cells to cells or tissues of their microenvironment. Other mobilization agents induce the release of proteases that cleave the adhesion molecules or support structures between stem cells and their sites of attachment. As used herein, the term “mobilization agent” refers to a wide range of molecules that act to enhance the mobilization of stem cells from their tissue or organ of residence, e.g., bone marrow (e.g., CD34+ stem cells) and spleen (e.g., Hox11+ stem cells), into peripheral blood. Mobilization agents include chemotherapeutic drugs, e.g., cyclophosphamide and cisplatin; cytokines, and chemokines, e.g., granulocyte colony-stimulating factor (G-CSF), granulocyte-macrophage colony-stimulating factor (GM-CSF), stem cell factor (SCF), Fms-related tyrosine kinase 3 (flt-3) ligand, stromal cell-derived factor 1 (SDF-1); agonists of the chemokine (C—C motif) receptor 1 (CCR1), such as chemokine (C—C motif) ligand 3 (CCL3, also known as macrophage inflammatory protein-1α (Mip-1α)); agonists of the chemokine (C—X—C motif) receptor 1 (CXCR1) and 2 (CXCR2), such as chemokine (C—X—C motif) ligand 2 (CXCL2) (also known as growth-related oncogene protein-β (Gro-β)), and CXCL8 (also known as interleukin-8 (IL-8)); agonists of CXCR4, such as CTCE-02142, and Met-SDF-1; Very Late Antigen (VLA)-4 inhibitors; antagonists of CXCR4, such as TG-0054, plerixafor (also known as AMD3100), and AMD3465, or any combination of the previous agents. A mobilization agent increases the number of stem cells in peripheral blood, thus allowing for a more accessible source of stem cells.

### Base Editing Enzymes:

In some embodiments, the base-editing enzyme of the present invention comprises a defective CRISPR/Cas nuclease. The sequence recognition mechanism is the same as for the non-defective CRISPR/Cas nuclease. Typically, the defective CRISPR/Cas nuclease of the invention comprises at least one RNA binding domain. The RNA binding domain interacts with a guide RNA molecule as defined hereinafter. However the defective CRISPR/Cas nuclease of the invention is a modified version with no nuclease activity. Accordingly, the defective CRISPR/Cas nuclease specifically recognizes the guide RNA molecule and thus guides the base-editing enzyme to its target DNA sequence.

In some embodiments, the defective CRISPR/Cas nuclease can be modified to increase nucleic acid binding affinity and/or specificity, alter an enzymatic activity, and/or change another property of the protein. In some embodiments, the nuclease domains of the protein can be modified, deleted, or inactivated. In some embodiments, the protein can be truncated to remove domains that are not essential for the function of the protein. In some embodiments, the protein is truncated or modified to optimize the activity of the RNA binding domain.

In some embodiments, the CRISPR/Cas nuclease consists of a mutant CRISPR/Cas nuclease i.e. a protein having one or more point mutations, insertions, deletions, truncations, a fusion protein, or a combination thereof. In some embodiments, the mutant has the RNA-guided DNA binding activity, but lacks one or both of its nuclease active sites. In some embodiments, the mutant comprises an amino acid sequence having at least 50% of identity with the wild type amino acid sequence of the CRISPR/Cas nuclease. Various CRISPR/Cas nucleases can be used in this invention. Non-limiting examples of suitable CRISPR/CRISPR/Cas nucleases include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966. See e.g., WO2014144761 WO2014144592, WO2013176772, US20140273226, and US20140273233, the contents of which are incorporated herein by reference in their entireties.

In some embodiments, the CRISPR/Cas nuclease is derived from a type II CRISPR-Cas system. In some embodiments, the CRISPR/Cas nuclease is derived from a Cas9 protein. The Cas9 protein can be from Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina, inter alia.

In some embodiments, the CRISPR/Cas nuclease is a mutant of a wild type CRISPR/Cas nuclease (such as Cas9) or a fragment thereof. In some embodiments, the CRISPR/Cas nuclease is a mutant Cas9 protein from S. pyogenes.

Methods for generating a Cas9 protein (or a fragment thereof) having an inactive DNA cleavage domain are known (See, e.g., Jinek et al., Science. 337:816-821 (2012); Qi et al., “Repurposing CRISPR as an RNA-Guided Platform for Sequence-Specific Control of Gene Expression” (2013) Cell. 28; 152 (5): 1173-83, the entire contents of each of which are incorporated herein by reference). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H841A completely inactivate the nuclease activity of S. pyogenes Cas9 (Jinek et al., Science. 337:816-821 (2012); Qi et al., Cell. 28; 152 (5): 1173-83 (2013).

In some embodiments, the CRISPR/Cas nuclease of the present invention is nickase and more particularly a Cas9 nickase i.e. the Cas9 from S. pyogenes having one mutation selected from the group consisting of D10A and H840A. In some embodiments, the nickase of the present invention comprises the amino acid sequence as set forth in SEQ ID NO: 2 or SEQ ID NO:3.

> S. pyogenes nCas9 Protein Sequence having the D10A
mutation
SEQ ID NO: 2
MDKKYSIGLAIGTNSVGWAVITDEYKVPSKKFKVLGNTDRHSIKKNLIGALLFDSGETAEATRLKRTAR
RRYTRRKNRICYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQLVQTYNQLFEENPINASGVD
AKAILSARLSKSRRLENLIAQLPGEKKNGLFGNLIALSLGLTPNFKSNFDLAEDAKLQLSKDTYDDDLD
NLLAQIGDQYADLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQRTFDNGSIPHQI
HLGELHAILRRQEDFYPFLKDNREKIEKILTFRIPYYVGPLARGNSRFAWMTRKSEETITPWNFEEVVD
KGASAQSFIERMTNFDKNLPNEKVLPKHSLLYEYFTVYNELTKVKYVTEGMRKPAFLSGEQKKAIVDLL
FKTNRKVTVKQLKEDYFKKIECFDSVEISGVEDRFNASLGTYHDLLKIIKDKDELDNEENEDILEDIVL
TLTLFEDREMIEERLKTYAHLFDDKVMKQLKRRRYTGWGRLSRKLINGIRDKQSGKTILDFLKSDGFAN
RNFMQLIHDDSLTFKEDIQKAQVSGQGDSLHEHIANLAGSPAIKKGILQTVKVVDELVKVMGRHKPENI
VIEMARENQTTQKGQKNSRERMKRIEEGIKELGSQILKEHPVENTQLQNEKLYLYYLQNGRDMYVDQEL
DINRLSDYDVDHIVPQSFLKDDSIDNKVLTRSDKNRGKSDNVPSEEVVKKMKNYWRQLLNAKLITQRKF
DNLTKAERGGLSELDKAGFIKRQLVETRQITKHVAQILDSRMNTKYDENDKLIREVKVITLKSKLVSDF
RKDFQFYKVREINNYHHAHDAYLNAVVGTALIKKYPKLESEFVYGDYKVYDVRKMIAKSEQEIGKATAK
YFFYSNIMNFFKTEITLANGEIRKRPLIETNGETGEIVWDKGRDFATVRKVLSMPQVNIVKKTEVQTGG
FSKESILPKRNSDKLIARKKDWDPKKYGGFDSPTVAYSVLVVAKVEKGKSKKLKSVKELLGITIMERSS
FEKNPIDFLEAKGYKEVKKDLIIKLPKYSLFELENGRKRMLASAGELQKGNELALPSKYVNFLYLASHY
EKLKGSPEDNEQKQLFVEQHKHYLDEIIEQISEFSKRVILADANLDKVLSAYNKHRDKPIREQAENIIH
LFTLTNLGAPAAFKYFDTTIDRKRYTSTKEVLDATLIHQSITGLYETRIDLSQLGGD
> S. pyogenes nCas9 Protein Sequence having the H840A
mutation
SEQ ID NO: 3
MDKKYSIGLDIGTNSVGWAVITDEYKVPSKKFKVLGNTDRHSIKKNLIGALLFDSGETAEATRLKRTAR
RRYTRRKNRICYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQLVQTYNQLFEENPINASGVD
AKAILSARLSKSRRLENLIAQLPGEKKNGLFGNLIALSLGLTPNFKSNFDLAEDAKLQLSKDTYDDDLD
NLLAQIGDQYADLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQRTFDNGSIPHQI
HLGELHAILRRQEDFYPFLKDNREKIEKILTFRIPYYVGPLARGNSRFAWMTRKSEETITPWNFEEVVD
KGASAQSFIERMTNFDKNLPNEKVLPKHSLLYEYFTVYNELTKVKYVTEGMRKPAFLSGEQKKAIVDLL
FKTNRKVTVKQLKEDYFKKIECFDSVEISGVEDRFNASLGTYHDLLKIIKDKDELDNEENEDILEDIVL
TLTLFEDREMIEERLKTYAHLFDDKVMKQLKRRRYTGWGRLSRKLINGIRDKQSGKTILDFLKSDGFAN
RNFMQLIHDDSLTFKEDIQKAQVSGQGDSLHEHIANLAGSPAIKKGILQTVKVVDELVKVMGRHKPENI
VIEMARENQTTQKGQKNSRERMKRIEEGIKELGSQILKEHPVENTQLQNEKLYLYYLQNGRDMYVDQEL
DINRLSDYDVDAIVPQSFLKDDSIDNKVLTRSDKNRGKSDNVPSEEVVKKMKNYWRQLLNAKLITQRKE
DNLTKAERGGLSELDKAGFIKRQLVETRQITKHVAQILDSRMNTKYDENDKLIREVKVITLKSKLVSDE
RKDFQFYKVREINNYHHAHDAYLNAVVGTALIKKYPKLESEFVYGDYKVYDVRKMIAKSEQEIGKATAK
YFFYSNIMNFFKTEITLANGEIRKRPLIETNGETGEIVWDKGRDFATVRKVLSMPQVNIVKKTEVQTGG
FSKESILPKRNSDKLIARKKDWDPKKYGGFDSPTVAYSVLVVAKVEKGKSKKLKSVKELLGITIMERSS
FEKNPIDFLEAKGYKEVKKDLIIKLPKYSLFELENGRKRMLASAGELQKGNELALPSKYVNFLYLASHY
EKLKGSPEDNEQKQLFVEQHKHYLDEIIEQISEFSKRVILADANLDKVLSAYNKHRDKPIREQAENIIH
LFTLTNLGAPAAFKYFDTTIDRKRYTSTKEVLDATLIHQSITGLYETRIDLSQLGGD

In some embodiments, the Cas9 variants having mutations other than D10A or H840A are used, which e.g., result in nuclease inactivated Cas9 (dCas9). Such mutations, by way of example, include other amino acid substitutions at D10 and H840, or other substitutions within the nuclease domains of Cas9 (e.g., substitutions in the HNH nuclease subdomain and/or the RuvC1 subdomain). In some embodiments, variants of dCas9 are provided which are at least about 70% identical, at least about 80% identical, at least about 90% identical, at least about 95% identical, at least about 98% identical, at least about 99% identical, at least about 99.5% identical, or at least about 99.9% to SEQ ID NO: 2 or 3. In some embodiments, variants of dCas9 are provided having amino acid sequences which are shorter, or longer than SEQ ID NO: 2 or 3, by about 5 amino acids, by about 10 amino acids, by about 15 amino acids, by about 20 amino acids, by about 25 amino acids, by about 30 amino acids, by about 40 amino acids, by about 50 amino acids, by about 75 amino acids, by about 100 amino acids or more.

According to the present invention, the second component of the base-editing enzyme herein disclosed comprises a non-nuclease DNA modifying enzyme that is a deaminase.

In some embodiments, the deaminase is an adenosine deaminase. In some embodiments, the deaminase is an ADAT family deaminase. In some embodiments, the adenosine deaminase variant is a TadA deaminase. In some embodiments, the adenosine deaminase variant is a Staphylococcus aureus TadA, a Bacillus subtilis TadA, a Salmonella typhimurium TadA, a Shewanella putrefaciens TadA, a Haemophilus influenzae F3031 TadA, a Caulobacter crescentus TadA, or a Geobacter sulfurreducens TadA, or a fragment thereof. In some embodiments, the TadA deaminase is an E. coli TadA deaminase (ecTadA). In some embodiments, the TadA deaminase is a truncated E. coli TadA deaminase. For example, the truncated ecTadA may be missing one or more N-terminal amino acids relative to a full-length ecTadA. In some embodiments, the truncated ecTadA may be missing 1, 2, 3, 4, 5, 6, 7, 8, 9, 10, 11, 12, 13, 14, 15, 6, 17, 18, 19, or 20 N-terminal amino acid residues relative to the full length ecTadA. In some embodiments, the truncated ecTadA may be missing 1, 2, 3, 4, 5,6, 7, 8, 9, 10, 11, 12, 13, 14, 15, 6, 17, 18, 19, or 20C-terminal amino acid residues relative to the full length ecTadA. In some embodiments, the TadA deaminase is TadA*7.10. In some embodiments, the TadA deaminase is a TadA*8 variant. For example, deaminase are described in International PCT Application WO2018/027078, WO2017/070632, WO/2020/168132, WO/2021/050571 each of which is incorporated herein by reference for its entirety. Also, see Komor, A. C., et al., “Programmable editing of a target base in genomic DNA without double-stranded DNA cleavage” Nature 533, 420-424 (2016); Gaudelli, N. M., et al., “Programmable base editing of A⋅T to G⋅C in genomic DNA without DNA cleavage” Nature 551, 464-471 (2017); Komor, A. C., et al., “Improved base excision repair inhibition and bacteriophage Mu Gam protein yields C: G-to-T: A base editors with higher efficiency and product purity” Science Advances 3: eaao4774 (2017)), and Rees, H. A., et al., “Base editing: precision chemistry on the genome and transcriptome of living cells.” Nat Rev Genet. 2018 December; 19 (12): 770-788. doi: 10.1038/s41576-018-0059-1, the entire contents of which are hereby incorporated by reference. An exemplary amino acid sequence for the wild type TadA (wt) adenosine deaminase is shown as SEQ ID NO: 4. In some embodiments, the amino acid sequence of the adenosine deaminase comprises at least 90% sequence identity to SEQ ID NO:4. In some embodiments, the amino acid sequence of the adenosine deaminase comprises the modification at position 82 as numbered in SEQ ID NO: 4. In some embodiments, the amino acid sequence comprises of the adenosine deaminase comprises a V82S modification, wherein position 82 is as numbered in SEQ ID NO: 4. In some embodiments, the amino acid sequence of the adenosine deaminase comprises the modification at position 166 as numbered in SEQ ID NO:4. In some embodiments, the amino acid sequence of the adenosine deaminase comprises a T166R modification, wherein position 166 is as numbered in SEQ ID NO: 4. In some embodiments, the amino acid sequence of the adenosine deaminase comprises modifications at positions 82 and 166 as numbered in SEQ ID NO: 4. In some embodiments, the amino acid sequence of the adenosine deaminase comprises V82S and T166R modifications, wherein positions 82 and 166 are as numbered in SEQ ID NO: 4. In some embodiments, the adenosine deaminase variant further comprises one or more of the following alterations: Y147T, Y147R, Q154S, Y123H, and Q154R. In some embodiments, the adenosine deaminase variant comprises a combination of alterations selected from the group consisting of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+I76Y; Y147R+Q154R+T166R; Y123H+Y147R+Q154R+I76Y; V82S+Y123H+Y147R+Q154R; and I76Y+V82S+Y123H+Y147R+Q154R. In some embodiments, the adenosine deaminase variant is TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, or TadA*8.24. In some embodiments, the adenosine deaminase is provided as a single (e.g., provided as a monomer) TadA variant as described above. In some embodiments, adenosine deaminase is provided as a heterodimer of a wild-type TadA (TadA (wt)) linked to a TadA variant as described above.

> TadA sequence
SEQ ID NO: 4
MSEVEFSHEYWMRHALTLAKRAWDEREVPVGAVLVHNNRVIGEGW
NRPIGRHDPTAHAEIMALRQGGLVMQNYRLIDATLYVTLEPCVMC
AGAMIHSRIGRVVFGARDAKTGAAGSLMDVLHHPGMNHRVEITEG
ILADECAALLSDFFRMRRQEIKAQKKAQSSTD

In some embodiments, the deaminase is fused to the N-terminus of the defective CRISPR/Cas nuclease. In some embodiments, the deaminase is fused to the C-terminus of the defective CRISPR/Cas nuclease. In some embodiments, the defective CRISPR/Cas nuclease and the deaminase are fused via a linker. In some embodiments, the linker comprises a (GGGGS)n (SEQ ID NO:5), a (G)n, an (EAAAK)n (SEQ ID NO: 6), a (GGS) n, an SGSETPGTSESATPES (SEQ ID NO: 7) motif (see, e.g., Guilinger J P, Thompson D B, Liu D R. Additional suitable linker motifs and linker configurations will be apparent to those of skill in the art. In some embodiments, suitable linker motifs and configurations include those described in Chen et al., Fusion protein linkers: property, design and functionality. Adv Drug Deliv Rev. 2013; 65 (10): 1357-69, the entire contents of which are incorporated herein by reference.

In some embodiments, the fusion protein may comprise additional features. Other exemplary features that may be present are localization sequences, such as nuclear localization sequences (NLS), cytoplasmic localization sequences, export sequences, such as nuclear export sequences, or other localization sequences, as well as sequence tags that are useful for solubilization, purification, or detection of the fusion proteins. Suitable localization signal sequences and sequences of protein tags are provided herein, and include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FLASH tags, V5 tags, and SBP-tags. Additional suitable features will be apparent to those of skill in the art.

Various base-editing enzymes are known in the art (see e.g. Improving cytidine and adenine base-editing enzymes by expression optimization and ancestral reconstruction. Nat Biotechnol. 2018 May 29) and typically include those described in Table A.

TABLE A
some exemplary base-editing enzymes

| Base-editing | |
|---|---|
| enzyme | References |
| ABEmax | Improving cytidine and adenine base-editing enzymes by expression |
| | optimization and ancestral reconstruction. Nat Biotechnol. 2018 May 29. |
| | pii: nbt.4172. doi: 10.1038/nbt.4172. |
| AncBE4max | Improving cytidine and adenine base-editing enzymes by expression |
| | optimization and ancestral reconstruction. Nat Biotechnol. 2018 May 29. |
| | pii: nbt.4172. doi: 10.1038/nbt.4172. |
| evoCDA1- | Continuous evolution of base-editing enzymes with expanded target |
| BE4max-NG | compatibility and improved activity. Nat Biotechnol. 2019 Jul. 22. pii: |
| | 10.1038/s41587-019-0193-0. doi: 10.1038/s41587-019-0193-0. |
| evoFERNY- | Continuous evolution of base-editing enzymes with expanded target |
| BE4max | compatibility and improved activity. Nat Biotechnol. 2019 Jul. 22. pii: |
| | 10.1038/s41587-019-0193-0. doi: 10.1038/s41587-019-0193-0. |
| CBE-NRCH | Miller SM, Wang T, Randolph PB, Arbab M, Shen MW, Huang TP, |
| | Matuszek Z, Newby GA, Rees HA, Liu DR. Continuous evolution of |
| | SpCas9 variants compatible with non-G PAMs. Nat Biotechnol. 2020 |
| | April; 38(4):471-481. doi: 10.1038/s41587-020-0412-8. Epub 2020 Feb. 10. |
| | PMID: 32042170; PMCID: PMC7145744. |
| CBE-SpG | Walton RT, Christie KA, Whittaker MN, Kleinstiver BP. Unconstrained |
| | genome targeting with near-PAMless engineered CRISPR-Cas9 variants. |
| | Science. 2020 Apr. 17; 368(6488):290-296. doi: 10.1126/science.aba8853. |
| | Epub 2020 Mar. 26. PMID: 32217751; PMCID: PMC7297043. |
| CBE-SpRY | Walton RT, Christie KA, Whittaker MN, Kleinstiver BP. Unconstrained |
| | genome targeting with near-PAMless engineered CRISPR-Cas9 variants. |
| | Science. 2020 Apr. 17;368(6488):290-296. doi: 10.1126/science.aba8853. |
| | Epub 2020 Mar. 26. PMID: 32217751; PMCID: PMC7297043. |
| ABE8e | Richter MF, Zhao KT, Eton E, Lapinaite A, Newby GA, Thuronyi BW, |
| | Wilson C, Koblan LW, Zeng J, Bauer DE, Doudna JA and Liu DR. Phage- |
| | assisted evolution of an adenine base editor with improved Cas domain |
| | compatibility and activity. Nat. Biotechnol. 2020 Mar. 38, 883-891. 1780 |
| | doi: 10.1038/s41587-020-0453-z |

### Guide RNA Molecules:

The second component of the gene-editing platform disclosed herein consists of a plurality of guide RNA molecules suitable for guiding the base-editing enzyme to a plurality of target sequences. In particular, the gene editing platform disclosed herein comprises i) at least one guide RNA molecule designed for guiding the base-editing enzyme to a target sequence of interest and ii) at least one guide RNA molecule designed for guiding the base-editing enzyme to a target sequence in the gene encoding for CD33 so as to knock-down the expression of CD33. In some embodiments, the gene editing platform disclosed herein thus comprises 2, 3, 4, 5, 6, 7, 8, 9, 10, 11, 12, 13, 14, 15, 16, 17, 18, or 20 guide RNA molecules.

In particular, the guide RNA molecules include a region that is complementary and capable of hybridization to a pre-selected target site of interest. In some embodiment, this guide sequence can comprise from about 10 nucleotides to more than about 25 nucleotides. For example, the region of base pairing between the guide sequence and the corresponding target site sequence can be about 10, 11, 12, 13, 14, 15, 16, 17, 18, 19, 20, 22, 23, 24, 25, or more than 25 nucleotides in length. In some embodiments, the guide sequence is about 17-20 nucleotides in length, such as 20 nucleotides.

Typically, a software program is used to identify candidate CRISPR target sequences on both strands of the DNA nucleic acid molecule containing the HBG genes based on desired guide sequence length and a CRISPR motif sequence (PAM) for a specified CRISPR enzyme. One requirement for selecting a suitable target nucleic acid is that it has a 3′ PAM site/sequence. Each target sequence and its corresponding PAM site/sequence are referred herein as a Cas-targeted site. Type II CRISPR system, one of the most well characterized systems, needs only Cas 9 protein and a guide RNA complementary to a target sequence to affect target cleavage. For example, target sites for Cas9 from S. pyogenes, with PAM sequences NGG, may be identified by searching for 5′-Nx-NGG-3′ both on the input sequence and on the reverse-complement of the input. Since multiple occurrences in the genome of the DNA target site may lead to nonspecific genome editing, after identifying all potential sites, the program filters out sequences based on the number of times they appear in the relevant reference genome. For those CRISPR enzymes for which sequence specificity is determined by a “seed” sequence, such as the 11-12 bp 5′ from the PAM sequence, including the PAM sequence itself, the filtering step may be based on the seed sequence. Thus, to avoid editing at additional genomic loci, results are filtered based on the number of occurrences of the seed: PAM sequence in the relevant genome. The user may be allowed to choose the length of the seed sequence. The user may also be allowed to specify the number of occurrences of the seed: PAM sequence in a genome for purposes of passing the filter. The default is to screen for unique sequences. Filtration level is altered by changing both the length of the seed sequence and the number of occurrences of the sequence in the genome. The program may in addition or alternatively provide the sequence of a guide sequence complementary to the reported target sequence(s) by providing the reverse complement of the identified target sequence(s). Further details of methods and algorithms to optimize sequence selection can be found in U.S. application Ser. No. 61/836,080; incorporated herein by reference.

In some embodiments, the gene editing platform of the present invention comprises at least one guide RNA molecule designed to target CD33 gene and that leads to start codon disruption when coupled with CBEs, or disrupts the splice donor site of exon1-2 when coupled either with CBEs or ABEs, and or disrupts the splice acceptor site of exon1-2 when coupled with ABEs.

Typically, the guide RNA molecule targets a nucleic acid sequence selected from Table 1.

In some embodiments, the gene editing platform comprises at least one guide RNA molecule that targets CD33 and that disrupts the splice donor site of exon1-2 when coupled either with CBEs or ABEs. In some embodiments, the guide RNA molecule targets the nucleic acid sequence as set forth in SEQ ID NO:13.

In some embodiments, the gene editing platform comprises (a) at least one adenine-base editor (ABE), and (b) a plurality of guide RNA molecules designed for guiding the base-editing enzyme(s) to a plurality of target sequences wherein at least one guide RNA molecule is designed for guiding the ABE to the target sequence as set forth in SEQ ID NO:13.

The guide RNA molecule of the present invention can be made by various methods known in the art including cell-based expression, in vitro transcription, and chemical synthesis. The ability to chemically synthesize relatively long RNAs (as long as 200 mers or more) using TC-RNA chemistry (see, e.g., U.S. Pat. No. 8,202,983) allows one to produce RNAs with special features that outperform those enabled by the basic four ribonucleotides (A, C, G and U). In particular, the RNA molecule of the present invention can be made with recombinant technology using a host cell system or an in vitro translation-transcription system known in the art. Details of such systems and technology can be found in e.g., WO2014144761 WO2014144592, WO2013176772, US20140273226, and US20140273233, the contents of which are incorporated herein by reference in their entireties.

In some embodiments, the guide RNA molecule may include one or more modifications. Such modifications may include inclusion of at least one non-naturally occurring nucleotide, or a modified nucleotide, or analogs thereof. Modified nucleotides may be modified at the ribose, phosphate, and/or base moiety. Modified nucleotides may include 2′-O-methyl analogs, 2′-deoxy analogs, or 2′-fluoro analogs. The nucleic acid backbone may be modified, for example, a phosphorothioate backbone may be used. The use of locked nucleic acids (LNA) or bridged nucleic acids (BNA) may also be possible. Further examples of modified bases include, but are not limited to, 2-aminopurine, 5-bromo-uridine, pseudouridine, inosine, 7-methylguanosine.

### Uses:

In some embodiments, the gene editing platform is used to alter a target polynucleotide sequence of interest in the eukaryotic cell for any purpose. In some embodiments, the target polynucleotide sequence of interest in the eukaryotic cell is altered to generate a mutate cell, which results in a genotype that differs from its original genotype. In some embodiments, the target polynucleotide sequence of interest in the eukaryotic cell is altered to correct or repair a genetic mutation (e.g., to restore a normal phenotype to the cell). In some embodiments, the target polynucleotide sequence of interest in the eukaryotic cell is altered to induce a genetic mutation (e.g., to disrupt the function of a gene or genomic element). In some embodiments, the alteration may be a homozygous alteration or a heterozygous alternation. In some embodiments, the alteration may be an insertion, deletion, or the combination thereof. As will be appreciated by those skilled in the art, an insertion/deletion in a coding region of a genomic sequence will result in a frameshift mutation or a premature stop codon. In some embodiments, the alteration may be a point mutation.

In some embodiments, the gene editing platform of the present invention is used to generate a knock-out of a target polynucleotide sequence. The knocking out of a selected polynucleotide sequence can be useful for many applications, such as knocking out a target polynucleotide sequence of interest in the eukaryotic cell clone in vitro for research purposes; and knocking out a target polynucleotide sequence ex vivo for treating or preventing a disorder associated with increased expression of the target polynucleotide sequence. As used herein, the term “knock out” includes deleting all or a portion of the target polynucleotide sequence in a way that mutes the function of the target polynucleotide sequence.

In some embodiments, the alternation may result in a change of the target polynucleotide sequence of interest from an undesired sequence to a desired sequence. In some embodiments, the gene editing platform of the present invention is used to correct any type of mutation or error in a target polynucleotide sequence of interest, including but not limited to inserting a nucleotide sequence that is missing from a target polynucleotide sequence due to a deletion, deleting a nucleotide sequence from a target polynucleotide sequence due to an insertion mutation, and replacing an incorrect nucleotide sequence with a correct nucleotide sequence.

In some embodiments, the alteration results in reduced or increased expression of a target polynucleotide sequence of interest.

In some embodiments, the gene editing platform is used for increasing the fetal hemoglobin content in the eukaryotic cell.

In some embodiments, the gene editing platform is used to edit the HBG1 or HBG2 promoter and subsequently increasing the expression of γ-globin.

In some embodiments, the gene editing platform is suitable for introducing some mutations in the HBG1 or HBG2 promoter so that at least one transcriptional activator binding site is introduced in said promoter. In some embodiments, the gene editing platform is particularly suitable for introducing a new transcriptional activator binding site for KLF1, TAL1 or GATA1. In some embodiments, the gene editing platform herein disclosed introduces the −198T>C mutation in the HBG1 or HBG2 promoter so that the KFL1 activator can now binds to the promoter. In some embodiments, the gene editing platform herein disclosed introduces the −175T>C mutation in the HBG1 or HBG2 promoter so that the TAL1 activator can now binds to the promoter. In some embodiments, the gene editing platform herein disclosed introduces the −113A>G mutation in the HBG1 or HBG2 promoter so that the GATA1 activator can now binds to the promoter. In some embodiments, the gene editing platform herein disclosed is particularly suitable for editing the −200 region in the HBG1 or HBG2 promoter so that the binding site for the LRF repressor is disrupted. In some embodiments, the gene editing platform herein disclosed introduces at least one mutation selected from the group consisting of −201C>T, −200C>T, −197C>T, −196C>T, −195C>T and −194C>T in the HBG1 or HBG2 promoter so that the binding site for the LRF repressor is disrupted. In some embodiments, the gene editing herein disclosed is particularly suitable for editing the −115 region in the HBG1 or HBG2 promoter so that the binding site for the BCL11A repressor is disrupted. In some embodiments, the gene editing platform herein disclosed introduces at least one mutation selected from the group consisting of −114C>T, −113C>T, −115C>T and −116C>T in the HBG1 or HBG2 promoter so that the binding site for the BCL11A repressor is disrupted.

In some embodiments, the gene editing platform is used to edit the +55-kb region of the erythroid-specific BCL11A enhancer, thereby disrupting the ATF4 binding site in said region so as to repress the expression of BCL11A and subsequently increase the expression of γ-globin.

In some embodiments, the gene editing platform is used to repress the expression of α-globin.

In some embodiments, the gene editing platform is used to edit the MCS-R2 region present in the locus control region of HBA1 and HBA2 genes, thereby editing said MCS-R2 region and subsequently repressing the expression of α-globin in said eukaryotic cell. In some embodiments, the gene editing platform is suitable for introducing some mutations in the MCS-R2 region so that at least one transcriptional activator binding site is disrupted in said region. In some embodiments, the gene editing platform is particularly suitable for disrupting at least one transcriptional activator binding site for GATA1 or NF-E2 in the MCS-R2 region.

In some embodiments, the gene editing platform is used to restore the normal expression of β-globin in a eukaryotic cell carrying the CD39 (CAG>TAG) mutation. In some embodiments, the gene editing platform comprises least one guide RNA molecule for guiding an adenine base-editor to at least one target sequence comprising the CD39 (CAG>TAG) mutation and thereby restoring the production of β-globin in the eukaryotic cell.

In some embodiments, the gene editing platform is used to restore the normal expression of β-globin in a eukaryotic cell carrying the IVS2-1 (G>A) mutation. In some embodiments, the gene editing platform comprises least one guide RNA molecule for guiding an adenine base-editor to at least one target sequence comprising the IVS2-1 (G>A) mutation and thereby restoring the production of β-globin in the eukaryotic cell.

In some embodiments, the gene editing platform is used to restore the normal expression of β-globin in a eukaryotic cell carrying the IVS1-110 (G>A) mutation. In some embodiments, the gene editing platform comprises least one guide RNA molecule for guiding an adenine base-editor to at least one target sequence comprising the IVS1-110 (G>A) mutation and thereby restoring the production of β-globin in the eukaryotic cell.

### Vectors:

In some embodiments, the different components of the gene editing platform of the present invention are provided to the eukaryotic cell through expression from one or more expression vectors. For example, the nucleic acids encoding the guide RNA molecule or the base-editing enzyme can be cloned into one or more vectors for introducing them into the eukaryotic cell. The vectors are typically prokaryotic vectors, e.g., plasmids, or shuttle vectors, or insect vectors, for storage or manipulation of the nucleic acid encoding the guide RNA molecule or the base-editing enzyme herein disclosed. Preferably, the nucleic acids are isolated and/or purified. Thus, the present invention provides recombinant constructs or vectors having sequences encoding one or more of the guide RNA molecule or base-editing enzymes described above. Examples of the constructs include a vector, such as a plasmid or viral vector, into which a nucleic acid sequence of the invention has been inserted, in a forward or reverse orientation. In some embodiments, the construct further includes regulatory sequences. A “regulatory sequence” includes promoters, enhancers, and other expression control elements (e.g., polyadenylation signals). Regulatory sequences include those that direct constitutive expression of a nucleotide sequence, as well as inducible regulatory sequences. The design of the expression vector can depend on such factors as the choice of the eukaryotic cell to be transformed, transfected, or infected, the desired expression level, and the like. Large numbers of suitable vectors and promoters are known to those of skill in the art, and are commercially available. Appropriate cloning and expression vectors for use with eukaryotic hosts are also described in e.g., Sambrook et al. (2001, Molecular Cloning: A Laboratory Manual, Cold Spring Harbor Press). The vector can be capable of autonomous replication or integration into a host DNA. The vector may also include appropriate sequences for amplifying expression. In addition, the expression vector preferably contains one or more selectable marker genes to provide a phenotypic trait for selection of transformed host cells such as dihydrofolate reductase or neomycin resistance for eukaryotic cell cultures, or such as tetracycline or ampicillin resistance in E. coli. Any of the procedures known in the art for introducing foreign nucleotide sequences into host cells may be used. Examples include the use of calcium phosphate transfection, polybrene, protoplast fusion, electroporation, nucleofection, liposomes, microinjection, naked DNA, plasmid vectors, viral vectors, both episomal and integrative, and any of the other well-known methods for introducing cloned genomic DNA, cDNA, synthetic DNA or other foreign genetic material into a host cell.

In some embodiments, the different components of the gene editing platform of the present invention are provided to the population of cells through the use of an RNA-encoded system. For instance, the base-editing system may be provided to the population of cells through the use of a chemically modified mRNA-encoded adenine or cytidine base editor together with modified guide RNA as described in Jiang, T., Henderson, J. M., Coote, K. et al. Chemical modifications of adenine base editor mRNA and guide RNA expand its application scope. Nat Commun 11, 1979 (2020). In particular, engineered RNA-encoded base-editing enzymes (e.g. ABE) system are prepared by introducing various chemical modifications to both mRNA that encoded the base-editing enzyme and guide RNA. In particular said modifications consist in uridine depleted mRNAs modified with 5-methoxyuridine: synonymous codons may be introduced to deplete uridines as much as possible without altering the coding sequence and replaced all the remaining uridines with 5-methoxyuridine. Said optimized base editing system exhibits higher editing efficiency at some genomic sites compared to DNA-encoded system. It is also possible to encapsulate the modified mRNA and guide RNA into lipid nanoparticle (LNP) for allowing lipid nanoparticle (LNP)-mediated delivery.

In some embodiments, the different components of the gene editing platform of the present invention are provided to the population of cells through the use of ribonucleoprotein (RNP) complexes. For instance the base-editing enzyme can be pre-complexed with one or more guide RNA molecules to form a ribonucleoprotein (RNP) complex. The RNP complex can thus be introduced into the eukaryotic cell. Introduction of the RNP complex can be timed. The cell can be synchronized with other cells at G1, S, and/or M phases of the cell cycle. RNP delivery avoids many of the pitfalls associated with mRNA, DNA, or viral delivery. Typically, the RNP complex is produced simply by mixing the proteins (i.e. the base-editing enzyme) and one or more guide RNA molecules in an appropriate buffer. This mixture is incubated for 5-10 min at room temperature before electroporation. Electroporation is a delivery technique in which an electrical field is applied to one or more cells in order to increase the permeability of the cell membrane. In some embodiments, genome editing efficiency can be improved by adding a transfection enhancer oligonucleotide.

In some embodiments, a plurality of successive transfections are performed for reaching a desired level of mutagenesis in the cell.

### Methods of Enrichment:

A further object of the present invention relates to a method of preparing a substantially pure population of edited eukaryotic cells comprising the steps of i) editing a population of eukaryotic cells by the editing method herein disclosed and ii) enriching the population of edited eukaryotic cells that is negative for CD33.

According to the present invention, CD33 is indeed used as a negative marker for enriching the edited eukaryotic cells. Furthermore, CD33⁻ selection of the edited cells leads to higher base-editing efficiencies and eliminates poorly edited cells that could outcompete edited cells in engrafting in the bone marrow.

Cell enrichment can be accomplished by any means known to one of ordinary skill in the art. In some embodiments, the method of enriching cells comprises flow cytometry, cell sorting, magnetic activated cell sorting (for example as commercially used in Miltenyi Biotec MACS Technology or Dynal magnetic bead selection), antibody panning and red-cell resetting. Other methods for enrichment are also contemplated by the present invention. According to the present invention, the method comprises selecting the cells that have reduced expression, or do not substantially express CD33 as a cell surface marker. For example, flow cytometry may be used to enrich for cells that negative for CD33. Accordingly, FACS can be used with the methods described herein to isolate and detect the population of cells of the present invention. FACS typically involves using a flow cytometer capable of simultaneous excitation and detection of multiple fluorophores, such as a BD Biosciences FACSCanto™ flow cytometer, used substantially according to the manufacturer's instructions. The cytometric systems may include a cytometric sample fluidic subsystem, as described below. In addition, the cytometric systems include a cytometer fluidically coupled to the cytometric sample fluidic subsystem. Systems of the present disclosure may include a number of additional components, such as data output devices, e.g., monitors, printers, and/or speakers, softwares (e.g. (Flowjo, Laluza . . . ), data input devices, e.g., interface ports, a mouse, a keyboard, etc., fluid handling components, power sources, etc. Typically, the population of cells is contacted with a panel of antibodies specific for the specific phenotypic markers of interest (CD33). Typically, the antibodies are labelled with a tag to facilitate the isolation and detection of population of cells of the interest. Suitable labels include fluorescent molecules, radioisotopes, nucleotide chromophores, enzymes, substrates, chemiluminescent moieties, magnetic particles, bioluminescent moieties, and the like. As such, a label is any composition detectable by spectroscopic, photochemical, biochemical, immunochemical, electrical, optical or chemical means. Non-limiting examples of fluorescent labels or tags for labeling the agents such as antibodies for use in the methods of invention include Hydroxycoumarin, Succinimidyl ester, Aminocoumarin, Succinimidyl ester, Methoxycoumarin, Succinimidyl ester, Cascade Blue, Hydrazide, Pacific Blue, Maleimide, Pacific Orange, Lucifer yellow, NBD, NBD-X, R-Phycoerythrin (PE), a PE-Cy5 conjugate (Cychrome, R670, Tri-Color, Quantum Red), a PE-Cy7 conjugate, Red 613, PE-Texas Red, PerCP, PerCPeFluor 710, PE-CF594, Peridinin chlorphyll protein, TruRed (PerCP-Cy5.5 conjugate), FluorX, Fluoresceinisothyocyanate (FITC), BODIPY-FL, TRITC, X-Rhodamine (XRITC), Lissamine Rhodamine B, Texas Red, Allophycocyanin (APC), an APC-Cy7 conjugate, Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, Alexa Fluor 750, Alexa Fluor 790, Cy2, Cy3, Cy3B, Cy3.5, Cy5, Cy5.5, Cy7, BV 785, BV711, BV421, BV605, BV510 or BV650. The aforementioned assays may involve the binding of the antibodies to a solid support. The solid surface could be a microtitration plate coated with the antibodies. Alternatively, the solid surfaces may be beads, such as activated beads, magnetically responsive beads. Beads may be made of different materials, including but not limited to glass, plastic, polystyrene, and acrylic. In addition, the beads are preferably fluorescently labelled. In some embodiments, fluorescent beads are those contained in TruCount™ tubes, available from Becton Dickinson Biosciences, (San Jose, California).

### Methods of Therapy:

A further object of the present invention relates to a method of therapy in a patient in need thereof, the method comprising transplanting a therapeutically effective amount of a population of edited eukaryotic cells (enriched or not for CD33− cells) obtained by the methods herein disclosed.

In particular, the method is particularly suitable for treating a β-hemoglobinopathy. In some embodiments, the β-hemoglobinopathy is a sickle cell disease. In some embodiments, the β-hemoglobinopathy is a β-thalassemia.

In some embodiments, the population of cell is autologous to the subject, meaning the population of cells is derived from the same subject.

In some embodiments, the methods of therapy as disclosed herein further comprise administering to the patient a therapeutically effective amount of an agent that is capable of depleting CD33 positive cells.

In some embodiments, the agent is an antibody that binds to CD33 and depletes CD33+ cells (i.e. a “depleting antibody”).

As used herein, the term “depletion” with respect to CD33+ cells, refers to a measurable decrease in the number of CD33+ cells in the subject. The reduction can be at least about 10%, e.g., at least about 20%, 30%, 40%, 50%, 60%, 70%, 80%, 90%, 95%, 96%, 97%, 98%, 99%, or more. In some embodiments, the depleting antibody binds to CD33.

In some embodiments, the depleting antibody mediates antibody-dependent cell-mediated cytotoxicity. As used herein the term “antibody-dependent cell-mediated cytotoxicity” or ‘ADCC’” refer to a cell-mediated reaction in which non-specific cytotoxic cells (e.g., Natural Killer (NK) cells, neutrophils, and macrophages) recognize bound antibody on a target cell and subsequently cause lysis of the target cell. In some embodiments, the depleting antibody is an IgG1 antibody. In some embodiments, the depleting antibody is an IgG3 antibody.

In some embodiments, the antibody suitable for depletion of CD33+ cells is conjugated to a therapeutic moiety, i.e. a drug.

In some embodiments, the therapeutic moiety can be, e.g., a cytotoxin, a chemotherapeutic agent, a cytokine, an immunosuppressant, an immune stimulator, a lytic peptide, or a radioisotope. Such conjugates are referred to herein as an “antibody-drug conjugates” or “ADCs”.

In some embodiments, the antibody suitable for depletion of CD33+ cells is conjugated to a cytotoxic moiety. The cytotoxic moiety may, for example, be selected from the group consisting of taxol; cytochalasin B; gramicidin D; ethidium bromide; emetine; mitomycin; etoposide; tenoposide; vincristine; vinblastine; colchicin; doxorubicin; daunorubicin; dihydroxy anthracin dione; a tubulin-inhibitor such as maytansine or an analog or derivative thereof; an antimitotic agent such as monomethyl auristatin E or F or an analog or derivative thereof; dolastatin 10 or 15 or an analogue thereof; irinotecan or an analogue thereof; mitoxantrone; mithramycin; actinomycin D; 1-dehydrotestosterone; a glucocorticoid; procaine; tetracaine; lidocaine; propranolol; puromycin; calicheamicin or an analog or derivative thereof; an antimetabolite such as methotrexate, 6 mercaptopurine, 6 thioguanine, cytarabine, fludarabin, 5 fluorouracil, decarbazine, hydroxyurea, asparaginase, gemcitabine, or cladribine; an alkylating agent such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C; a platinum derivative such as cisplatin or carboplatin; duocarmycin A, duocarmycin SA, rachelmycin (CC-1065), or an analog or derivative thereof; an antibiotic such as dactinomycin, bleomycin, daunorubicin, doxorubicin, idarubicin, mithramycin, mitomycin, mitoxantrone, plicamycin, anthramycin (AMC)); pyrrolo[2,1-c][1,4]-benzodiazepines (PDB); diphtheria toxin and related molecules such as diphtheria A chain and active fragments thereof and hybrid molecules, ricin toxin such as ricin A or a deglycosylated ricin A chain toxin, cholera toxin, a Shiga-like toxin such as SLT I, SLT II, SLT IIV, LT toxin, C3 toxin, Shiga toxin, pertussis toxin, tetanus toxin, soybean Bowman-Birk protease inhibitor, Pseudomonas exotoxin, alorin, saporin, modeccin, gelanin, abrin A chain, modeccin A chain, alpha-sarcin, Aleurites fordii proteins, dianthin proteins, Phytolacca americana proteins such as PAPI, PAPII, and PAP-S, Momordica charantia inhibitor, curcin, crotin, Sapaonaria officinalis inhibitor, gelonin, mitogellin, restrictocin, phenomycin, and enomycin toxins; ribonuclease (RNase); DNase I, Staphylococcal enterotoxin A; pokeweed antiviral protein; diphtherin toxin; and Pseudomonas endotoxin.

In some embodiments, the antibody suitable for depletion of CD33+ cells is conjugated to an auristatin or a peptide analog, derivative or prodrug thereof. Auristatins have been shown to interfere with microtubule dynamics, GTP hydrolysis and nuclear and cellular division (Woyke et al (2001) Antimicrob. Agents and Chemother. 45 (12): 3580-3584) and have anti-cancer (U.S. Pat. No. 5,663,149) and antifungal activity (Pettit et al., (1998) Antimicrob. Agents and Chemother. 42:2961-2965. For example, auristatin E can be reacted with para-acetyl benzoic acid or benzoylvaleric acid to produce AEB and AEVB, respectively. Other typical auristatin derivatives include AFP, MMAF (monomethyl auristatin F), and MMAE (monomethyl auristatin E). Suitable auristatins and auristatin analogs, derivatives and prodrugs, as well as suitable linkers for conjugation of auristatins to Abs, are described in, e.g., U.S. Pat. Nos. 5,635,483, 5,780,588 and 6,214,345 and in International patent application publications WO02088172, WO2004010957, WO2005081711, WO2005084390, WO2006132670, WO03026577, WO200700860, WO207011968 and WO205082023.

In some embodiments, the antibody suitable for depletion of CD33+ cells is Gemtuzumab ozogamicin that is a recombinant humanized IgG4 kappa antibody, which is conjugated with calicheamicin derivative, a cytotoxic antitumor antibiotic. In particular, Gemtuzumab comprises the light chain as set fort in SEQ ID NO:8 and the heavy chain as set forth in SEQ ID NO: 9.

>Light Chain
SEQ ID NO: 8
DIQLTQSPSTLSASVGDRVTITCRASESLDNYGIRFLTWFQQKPG
KAPKLLMYAASNQGSGVPSRFSGSGSGTEFTLTISSLQPDDFATY
YCQQTKEVPWSFGQGTKVEVKRTVAAPSVEIFPPSDEQLKSGTAS
VVCLLNNFYPREAKVQWKVDNALQSGNSQESVTEQDSKDSTYSLS
STLTLSKADYEKHKVYACEVTHQGLSSPVTKSENRGEC
>Heavy Chain
SEQ ID NO: 9
EVQLVQSGAEVKKPGSSVKVSCKASGYTITDSNIHWVRQAPGQSL
EWIGYIYPYNGGTDYNQKFKNRATLTVDNPTNTAYMELSSLRSED
TAFYYCVNGNPWLAYWGQGTLVTVSSASTKGPSVFPLAPCSRSTS
ESTAALGCLVKDYFPEPVTVSWNSGALTSGVHTFPAVLQSSGLYS
LSSVVTVPSSSLGTKTYTCNVDHKPSNTKVDKRVESKYGPPCPPC
PAPEFLGGPSVFLFPPKPKDTLMISRTPEVTCVVVDVSQEDPEVQ
FNWYVDGVEVHNAKTKPREEQFNSTYRVVSVLTVLHQDWLNGKEY
KCKVSNKGLPSSIEKTISKAKGQPREPQVYTLPPSQEEMTKNQVS
LTCLVKGFYPSDIAVEWESNGQPENNYKTTPPVLDSDGSFFLYSR
LTVDKSRWQEGNVESCSVMHEALHNHYTQKSLSLSLGK

### Kits

This invention further provides kits containing reagents for performing the above-described methods, including all component of the gene editing platform as disclosed herein for performing mutagenesis. To that end, one or more of the reaction components, e.g., guide RNA molecules, and nucleic acid molecules encoding for the base-editing enzymes for the methods disclosed herein can be supplied in the form of a kit for use. In some embodiments, the kit comprises (a) at least one base-editing enzyme a polynucleotide encoding thereof, and (b) a plurality of guide RNA molecules designed for guiding the base-editing enzyme(s) to a plurality of target sequences wherein at least one guide RNA molecule is designed for guiding the base-editing enzyme(s) to a target sequence in the gene encoding for CD33 so as to knock-down the expression of CD33. More particularly, the kit comprises (a) at least one adenine-base editor (ABE) a polynucleotide encoding thereof, and (b) a plurality of guide RNA molecules designed for guiding the base-editing enzyme(s) to a plurality of target sequences wherein at least one guide RNA molecule is designed for guiding the ABE to the target sequence as set forth in SEQ ID NO: 13. In some embodiments, the kit can include one or more other reaction components. In some embodiments, an appropriate amount of one or more reaction components is provided in one or more containers or held on a substrate. Examples of additional components of the kits include, but are not limited to, one or more host cells, one or more reagents for introducing foreign nucleotide sequences into host cells, one or more reagents (e.g., probes or PCR primers) for detecting expression of the guide RNA or base-editing enzymes or verifying the target nucleic acid's status, and buffers or culture media for the reactions. The kit may also include one or more of the following components: supports, terminating, modifying or digestion reagents, osmolytes, and an apparatus for detection. The components used can be provided in a variety of forms. For example, the components (e.g., enzymes, RNAs, probes and/or primers) can be suspended in an aqueous solution or as a freeze-dried or lyophilized powder, pellet, or bead. In the latter case, the components, when reconstituted, form a complete mixture of components for use in an assay. The kits of the invention can be provided at any suitable temperature. For example, for storage of kits containing protein components or complexes thereof in a liquid, it is preferred that they are provided and maintained below 0° C., preferably at or below −20° C., or otherwise in a frozen state. The kits can also include packaging materials for holding the container or combination of containers. Typical packaging materials for such kits and systems include solid matrices (e.g., glass, plastic, paper, foil, micro-particles and the like) that hold the reaction components or detection probes in any of a variety of configurations (e.g., in a vial, microtiter plate well, microarray, and the like). The kits may further include instructions recorded in a tangible form for use of the components.

The invention will be further illustrated by the following figures and examples. However, these examples and figures should not be interpreted in any way as limiting the scope of the present invention.

### FIGURES

FIG. 1. Base editing-mediated CD33 knockout in K562 cells.

A. Schematic representation of CD33 on chromosome 19 (chr19). Grey boxes indicate exons 1 and 2, and black boxes indicate the ATG start codon, the splice donor site (SD) of exon 1-2 and the splice acceptor site (SA) of exon 1-2. Black arrows depict sgRNAs designed to target CD33 with the target protospacer, and ATG, SD and SA targets highlighted in bold.

B. Experimental protocol used for base editing experiments using BE- and sgRNA-expressing plasmids in K562 cells.

C. A-T to G-C and C-G to T-A base-editing efficiency, calculated by the EditR software, in samples subjected to Sanger sequencing. Data are expressed as mean±SEM (n=3 biologically independent experiments).

D. Frequency of CD33⁺-cells in control (mock samples transfected with TE buffer) and edited samples, calculated by flow cytometry 5 days after treatment. The base editing efficiency (BE %) is presented below each condition. Data are expressed as mean±SEM (n=3 biologically independent experiments).

FIG. 2. CD33⁻ K562 cell selection eliminates non-edited cells.

A. Experimental protocol used for multiplex base editing experiments using BE- and sgRNA-expressing plasmids in K562 cells.

B. A-T to G-C and C-G to T-A base-editing efficiency at the HBG promoters, calculated by the EditR software, in samples transfected with 1 (single) or 2 (double) sgRNAs and subjected to Sanger sequencing. The base editing efficiency at CD33 (CD33 BE %) is presented below each condition. Data are expressed as mean±SEM (n=3 biologically independent experiments).

C. Frequency of CD33⁺-cells in control (mock samples transfected with TE buffer) and edited samples, calculated by flow cytometry 5 days after treatment. Data are expressed as mean±SEM (n=3 biologically independent experiments). *p≤0.05; **p≤0.0001 (One-way ANOVA).

D. A-T to G-C and C-G to T-A base-editing efficiency at CD33 and at the HBG promoters, calculated by the EditR software, in samples transfected with 2 sgRNAs, sorted for CD33 expression and subjected to Sanger sequencing. Data are expressed as mean±SEM (n=3 biologically independent experiments). **p≤0.01; ***p≤0.001; ****p≤0.0001 (Two-way ANOVA).

FIG. 3. CD33⁻ cell sorting leads to high BE efficiencies in SCD HSPCs.

A. Experimental protocol used for multiplex base editing experiments using BE mRNA and chemically modified sgRNA in SCD HSPCs.

B-C. Frequency of CD34⁺ cells for control (mock samples transfected with TE buffer) and single (ABE-KLF1 and CBE-LRF) or double (ABE-KLF1-CD33 and CBE-LRF-CD33) edited samples, calculated by flow cytometry 1 (B) and 2 (C) days after treatment. Data are expressed as single values (n=1 donor).

D-E. Frequency of CD33⁺-cells for control (mock samples transfected with TE buffer) and single (ABE-KLF1 and CBE-LRF) or double (ABE-KLF1-CD33 and CBE-LRF-CD33) edited samples, calculated by flow cytometry 1 (D) and 2 (E) days after treatment. Data are expressed as single values (n=1 donor).

F. CFC frequency for control (mock samples transfected with TE buffer), single (ABE-KLF1 and CBE-LRF) or double (ABE-KLF1-CD33 and CBE-LRF-CD33) edited samples, and double edited and sorted for CD33 expression [ABE-KLF1-CD33(CD33−) and CBE-LRF-CD33 (CD33−)] edited samples. Data are expressed as single values (n=1 donor).

G. A-T to G-C and C-G to T-A base-editing efficiency at CD33 and the HBG promoters, calculated by the EditR software, in samples transfected with 2 sgRNAs, sorted for CD33 expression and subjected to Sanger sequencing. Data are expressed as single values (n=1 donor).

### EXAMPLE

### Material & Methods

### Cell Line Culture

Human erythroleukemia K562 cells were maintained in RPMI 1640 (Lonza) containing glutamine and supplemented with 10% fetal bovine serum (Lonza), 2 mM Hepes (Life Technologies), 100 nM sodium pyruvate (Life Technologies), and penicillin and streptomycin (Life Technologies).

### HSPC Purification and Culture

We obtained human non-mobilized peripheral blood CD34+ HSPCs from SCD patients. SCD samples eligible for research purposes were obtained from the “Hôpital Necker-Enfants malades” Hospital (Paris, France). Written informed consent was obtained from all adult subjects. All experiments were performed in accordance with the Declaration of Helsinki. The study was approved by the regional investigational review board (reference: DC 2014-2272, CPP Ile-de-France II “Hôpital Necker-Enfants malades”). HSPCs were purified by immunomagnetic selection with MACS columns (Miltenyi Biotec) after immunostaining with the CD34 MicroBead Kit (Miltenyi Biotec). Twenty-four hours before transfection, CD34⁺ cells were thawed and cultured at a concentration of 5×10⁵ cells/ml in the “HSPC medium” containing StemSpan (STEMCELL Technologies) supplemented with penicillin/streptomycin (Gibco), 250 nM StemRegenin1 (STEMCELL Technologies), 38 nM UM171 (STEMCELL Technologies), and the following recombinant human cytokines (PeproTech): human stem cell factor (SCF) (300 ng/ml), Flt-3L (300 ng/ml), thrombopoietin (TPO) (100 ng/ml), and interleukin-3 (IL-3) (60 ng/ml).

### Plasmids

Plasmids used in this study include pCMV_ABEmax_P2A_GFP (Addgene #112101), ABEmax-OPT [generated by uridine depletion of the coding sequence of the pCMV_ABEmax_P2A_GFP (Addgene #112101) plasmid and by addition of a DNA fragment containing two copies of the 3′ untranslated region (UTR) of the HBB gene and a poly-A sequence of 96 adenines] and CBE-SpRY-OPT225.

sgRNA Design

We previously designed sgRNAs targeting the −200 region of the HBG1/2 promoters²⁵, and we manually designed sgRNAs targeting the CD33 gene (Table 1). To generate the sgRNA expression plasmid, oligonucleotides were annealed to create the sgRNA protospacer and the duplexes were ligated into the Bbs I-digested MA128 plasmid (provided by M. Amendola, Genethon, France). For RNA-mediated base editing we used chemically modified synthetic sgRNAs harboring 2′-O-methyl analogs and 3′-phosphorothioate nonhydrolyzable linkages at the first three 5′ and 3′ nucleotides (Synthego).

TABLE 1
sgRNA target sequences.

| | Target sequence | | |
|---|---|---|---|
| sgRNA | (5′ to 3′) | Position (hg19) | Strand |
| KLF1_bs_1 | GTGGGGAAGGGGCCCCCAAG | chr11: 5271279-5271298 (HBG1) | + |
| | (SEQ ID NO: 10) | chr11: 5276203-5276222 (HBG2) | |
| LRF_bs_2 | GCCCCTTCCCCACACTATCT | chr11: 5271272-5271291 (HBG1) | − |
| | (SEQ ID NO: 11) | chr11: 5276196-5276215 (HBG2) | |
| CD33-1 | AGCGGCATGTCTGAGGAAGC | chr19: 51728363-51728382 | − |
| | (SEQ ID NO: 12) | | |
| CD33-2 | CACTCACCTGCCCACAGCAG | chr19: 51728399-51728418 | − |
| | (SEQ ID NO: 13) | | |
| CD33-3 | CCCCACAGGGGCCCTGGCTA | chr19: 51728466-51728485 | + |
| | (SEQ ID NO: 14) | | |

mRNA In Vitro Transcription 10 μg of BE-expressing plasmids were digested overnight with 20 Units of a restriction enzyme that cleaves once after the poly-A tail or after the stop codon, for constructs with or without a poly-A tail, respectively. The linearized plasmids were purified using a PCR purification kit (QIAGEN) and were eluted in 30 μl of DNase/RNase-free water. 1 μg of linearized plasmid was used as template for the in vitro transcription (ivt) reaction (MEGAscript, Ambion). The ivt protocol was modified as follows. The GTP nucleotide solution was used at a final concentration of 3.0 mM instead of 7.5 mM and the anti-reverse cap analog N7-Methyl-3′-O-Methyl-Guanosine-5′-Triphosphate-5′-Guanosine (ARCA, Trilink) was used at a final concentration of 12.0 mM resulting in a final ratio of Cap:GTP of 4:1 that allows efficient capping of the mRNA. The incubation time for the ivt reaction was reduced to 30 minutes. For constructs without a poly-A tail already included in the plasmid, an additional step of polyadenylation was performed using manufacturer's guidelines (Poly-A tailing kit, Ambion). mRNA was precipitated using lithium chloride and resuspended in TE buffer in a final volume that allowed to achieve a concentration of >1 μg/μl. The mRNA quality was evaluated using Bioanalyzer (Agilent). CBE-SpRY-OPT2 mRNA, containing 5-methoxyUTP, capped with Cap1 analog, and subjected to silica membrane purification, was purchased from Trilink.

### Plasmid Transfection

K562 cells (10⁶ cells/condition) were transfected with 3.6 μg of a base editor-expressing plasmid and 1.2 μg of a sgRNA-containing plasmid or 2.4 μg of 2 sgRNA-containing plasmids. Cells transfected with TE buffer served as negative controls. We used the AMAXA Cell Line Nucleofector Kit V (VCA-1003) and the U-16 program (Nucleofector II). 18 h after transfection, transfection efficiency was evaluated by flow cytometry, using the Gallios (Beckman coulter) flow cytometer.

### RNA Transfection

0.5×10⁵ to 2×10⁵ CD34⁺ HSPCs per condition were transfected with 3.0 μg of the enzyme encoding mRNA, and one or two synthetic sgRNAs at a final concentration of 1.15 μM each. We used the P3 Primary Cell 4D-Nucleofector X Kit S (Lonza) and the CA137 program (Nucleofector 4D). Cells were incubated at 37° C. for 10 minutes before being transferred from the nucleocuvette strip well to a 96-well plate, upon addition of 80 μl of pre-warmed “HSPC medium”. Cells were incubated at 37° C. post-transfection. Cells transfected with TE buffer served as negative controls.

### Colony-Forming Cell (CFC) Assay

CD34⁺ HSPCs were plated at a concentration of 500 cells/mL in a methylcellulose-based medium (GFH4435, Stem Cell Technologies) under conditions supporting erythroid and granulocyte/monocyte differentiation. BFU-E and CFU-GM colonies were counted after 14 days. Colonies were randomly picked and collected as bulk populations (containing at least 25 colonies) to evaluate base-editing efficiency.

### Evaluation of Editing Efficiency

The base editing efficiency and the insertion and deletion (InDel) frequency were evaluated in K562 cells 3 days post-transfection, in “HSPC medium”-expanded cells 6 days post-transfection, and in BFU-E and CFU-GM 14 days after plating.

Genomic DNA was extracted from control and edited cells using PURE LINK Genomic DNA Mini kit (LifeTechnologies), or Quick-DNA/RNA Miniprep (ZYMO Research), following manufacturer's instructions. To evaluate the base editing efficiency at sgRNA target sites, we performed PCR followed by Sanger sequencing and EditR analysis (EditR: A Method to Quantify Base Editing from Sanger Sequencing)³³. TIDE analysis (Tracking of InDels by Decomposition) was also performed in order to evaluate the percentage of InDels in base edited samples³⁴

TABLE 2
Primers used to detect base-
editing and InDels events.

| Amplified region | F/R | Sequence (5′ to 3′) |
|---|---|---|
| HBG1 + HBG2 | F | AAAAACGGCTGACAA |
| promoters | | AAGAAGTCCTGGTAT |
| | | (SEQ ID NO: 15) |
| | R | ATAACCTCAGACGTT |
| | | CCAGAAGCGAGTGTG |
| | | (SEQ ID NO: 16) |
| CD33 | F | CAATCTGTGTGGAGGGGACAA |
| | | (SEQ ID NO: 17) |
| | R | AACTGGGGAGTTCTTGTCGT |
| | | (SEQ ID NO: 18) |

F, forward primer; R, reverse primer.

### Flow Cytometry Analysis

Flow cytometry analysis of CD33 surface marker for K562 cells and CD34⁺ HSPCs was performed using APC-conjugated anti-CD33 (551378, BD). Flow cytometry analyses were performed using Gallios (Beckman coulter) flow cytometer. Data were analyzed using FlowJo (BD Biosciences) software.

### Fluorescence-Activated Cell Sorting

K562 cells were transfected as described above and plated at a concentration of 5×10⁵ cells/ml. Five days after transfection, cells were stained with APC-conjugated anti-CD33 (551378, BD) and sorted using SH800 (Sony). Sorted and unsorted populations were collected for DNA extraction. CD34⁺ HSPCs were transfected 1 day after thawing as described above and plated at a concentration of 5×10⁵ cells/ml in “HSPC medium”. Two days after transfection, cells were stained with APC-conjugated anti-CD33 (551378, BD) and PE-conjugated anti-CD34 (550761, BD), and sorted using SH800 (Sony). Sorted and unsorted populations were either cultured at a concentration of 5×10⁵ cells/ml in “HSPC medium” for 6 days before collection for DNA extraction or plated for a CFC assay.

### Results:

### Base Editing-Mediated CD33 Knockout in K562 Cells

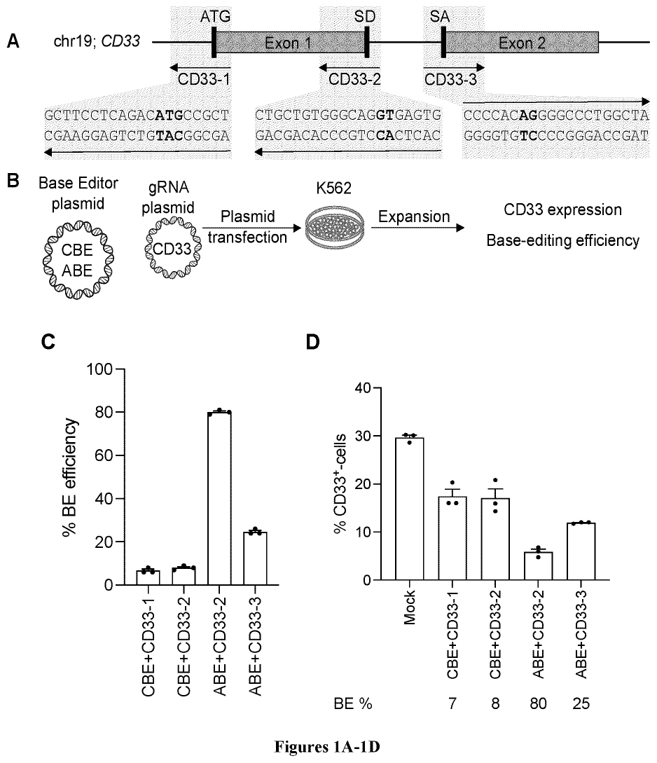

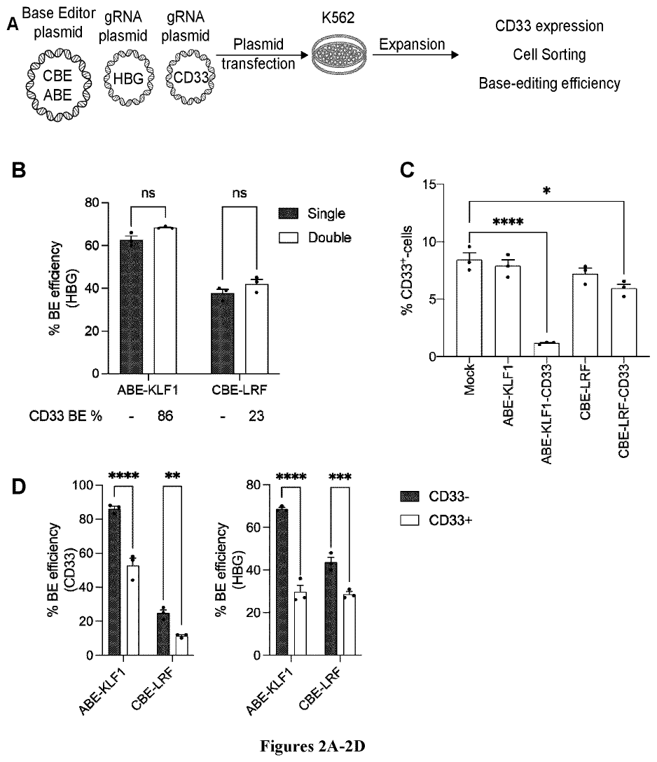

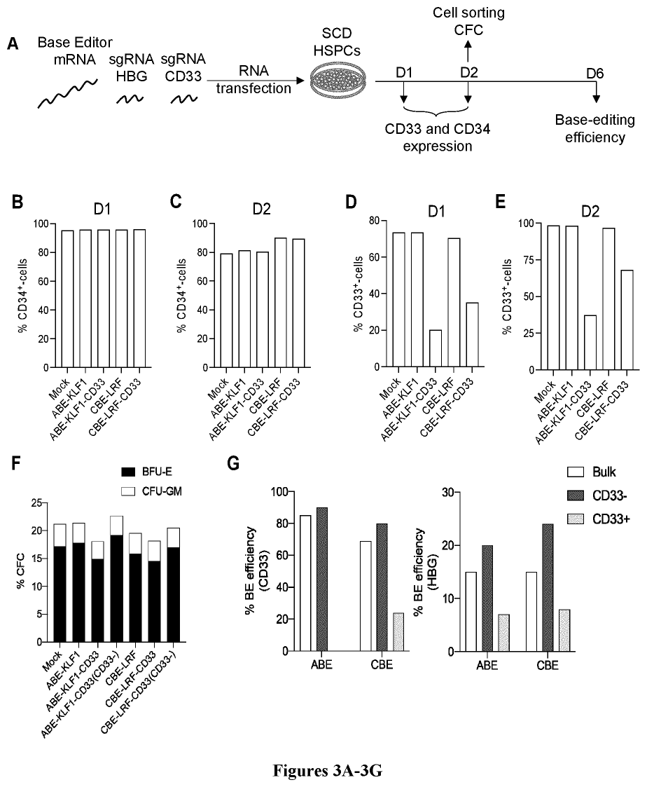

The BE system allows the generation of gene knockout (KO) through the introduction of point mutations. CBEs mediate gene KO via the insertion of stop codons, while both CBEs and ABEs alter gene expression through the disruption of either the ATG start codon, or the splice/acceptor donor sites. We designed sgRNAs that target CD33 (CD33-1, CD33-2 and CD33-3) and when coupled with CBEs or ABEs mediate CD33 KO. CD33-1 sgRNA leads to start codon disruption when coupled with CBEs, CD33-2 sgRNA disrupts the splice donor site of exon1-2 when coupled either with CBEs or ABEs, and CD33-3 sgRNA disrupts the splice acceptor site of exon1-2 when coupled with ABEs (FIG. 1A). We transfected K562 hematopoietic cells with a plasmid encoding for CBEs or ABEs (CBE-SpRY-OPT2 or ABEmax) and a sgRNA-encoding plasmid (FIG. 1B) and this led to different BE efficiencies varying from 7.0-80.0% (FIG. 1C). Flow cytometry analysis showed CD33 downregulation at protein level (29.6% and 17.4-5.8% in control and edited samples respectively; FIG. 1D). CD33-2 sgRNA and ABE was the best-performing combination to edit and KO CD33. Overall, these data indicate that BE efficiently downregulates CD33 expression in hematopoietic cell lines.

### CD33⁻ K562 Cell Selection Eliminates Non-Edited Cells

The DSB-free nature of BE allows safe multiplex editing without generating genomic rearrangements³². To evaluate the potential of enriching for cells edited at the HBG promoters through the selection of CD33− cells, we performed multiplex BE targeting of the HBG promoters and the CD33 gene simultaneously (FIG. 2A). We used two different BE systems to target the HBG promoters that efficiently reactivate HbF: LRF_bs_2 sgRNA coupled with CBE-SpRY-OPT2 to disrupt the LRF repressor BS and KLF1_bs_1 sgRNA coupled with ABEmax to generate the KLF1 activator BS25. For CD33 targeting, we selected CD33-2 sgRNA that when coupled with ABEmax gave the highest BE efficiency (80.0%) and a strong CD33 downregulation (FIGS. 1 C and D). We selected the same sgRNA coupled with CBE-SpRY-OPT2 as it led to CD33 downregulation despite the low BE efficiency (FIGS. 1 C and D). We transfected K562 cells with a plasmid encoding for CBEs or ABEs (CBE-SpRY-OPT2 or ABEmax) and either a sgRNA-encoding plasmid targeting the HBG promoters (single editing), or two sgRNA-encoding plasmids targeting the HBG promoters and the CD33 gene, respectively (double editing; FIG. 2A). We observed no statistically significant difference in HBG editing efficiency between single and double editing conditions (FIG. 2B). When targeting CD33, we observed a strong CD33 downregulation in ABE-treated samples and a mild reduction in CD33 expression in CBE-treated samples (FIG. 2C). We then sorted CD33⁻ and CD33⁺ populations. As expected, the frequency of edited CD33 alleles was higher in the CD33⁻ fraction (FIG. 2D). Interestingly, we observed higher BE efficiencies at the HBG promoters in the CD33⁻ population as compared to the CD33⁺ population (FIG. 2D), thus indicating that CD33⁻ cell sorting leads to elimination of poorly edited cells.

### CD33⁻ Cell Sorting Leads to Higher BE Efficiencies in SCD HSPCs

We applied the ABE or CBE double editing strategy in HSPCs obtained from one SCD patient. We used a clinically relevant protocol to deliver the BE system in SCD HSPC based on RNA transfection (mRNA encoding for a base editor and chemically modified synthetic sgRNAs; FIG. 3A)²⁵. SCD HSPCs were transfected with a mRNA encoding for ABEs or CBEs (ABEmax or CBE-SpRY-OPT2) and either a sgRNA targeting the HBG promoters (single editing), or 2 sgRNAs targeting the HBG promoters and the CD33 gene (double editing; FIG. 3A). One and two days after transfection, we evaluated the CD33 and CD34 expression in control and edited populations. At both time points, most of the cells were CD34⁺ (FIGS. 3B and C) and edited cells showed a decrease in CD33 expression (FIGS. 3D and E). Two days after transfection, we sorted CD33⁻ and CD33⁺ populations. Sorted and unsorted populations were either expanded in liquid culture (“HSPC medium”) or plated on a semi-solid medium that allows erythroid and granulocyte/monocyte differentiation of single progenitors (CFC assay; FIG. 3A). The CFC assay revealed no difference in the progenitors' viability between single and double edited cells, and between bulk and CD33⁻ sorted populations (FIG. 3F). Through RNA delivery, CBE was overall more efficient in HSPCs than in K562 (FIG. 3G), as previously observed²⁵. As expected, we observed no or lower BE at CD33 in CD33⁺ populations as compared to CD33⁻ populations. Importantly, we detected higher BE efficiencies in CD33⁻ populations than in the bulk or the CD33⁺ populations at HBG promoters for both ABE and CBE strategies (FIG. 3G). In conclusion, these results indicate that multiplex editing of HBG promoters and CD33, in conjunction with CD33⁻ selection of SCD HSPCs leads to higher BE efficiencies and eliminates poorly edited cells that could outcompete edited cells in engrafting in the bone marrow.

### REFERENCES

Throughout this application, various references describe the state of the art to which this invention pertains. The disclosures of these references are hereby incorporated by reference into the present disclosure.
1. Kato, G. J. et al. Sickle cell disease. Nat. Rev. Dis. Primer 4, 1-22 (2018).
2. Piel, F. B., Steinberg, M. H. & Rees, D. C. Sickle Cell Disease. N. Engl. J. Med. 376, 1561-1573 (2017).
3. Weatherall, D. J. Phenotype-genotype relationships in monogenic disease: lessons from the thalassaemias. Nat. Rev. Genet. 2, 245-255 (2001).
4. Cappellini, M. D., Porter, J. B., Viprakasit, V. & Taher, A. T. A paradigm shift on beta-thalassaemia treatment: How will we manage this old disease with new therapies? Blood Rev. 32, 300-311 (2018).
5. Taher, A. T., Weatherall, D. J. & Cappellini, M. D. Thalassaemia. The Lancet 391, 155-167 (2018).
6. Cavazzana, M., Antoniani, C. & Miccio, A. Gene Therapy for β-Hemoglobinopathies. Mol. Ther. J. Am. Soc. Gene Ther. 25, 1142-1154 (2017).
7. Forget, B. G. Molecular basis of hereditary persistence of fetal hemoglobin. Ann. N. Y. Acad. Sci. 850, 38-44 (1998).
8. Wienert, B. et al. Editing the genome to introduce a beneficial naturally occurring mutation associated with increased fetal globin. Nat. Commun. 6, 7085 (2015).
9. Wienert, B. et al. KLF1 drives the expression of fetal hemoglobin in British HPFH. Blood 130, 803-807 (2017).
10. Martyn, G. E. et al. A natural regulatory mutation in the proximal promoter elevates fetal globin expression by creating a de novo GATA1 site. Blood 133, 852-856 (2019).
11. Martyn, G. E., Quinlan, K. G. R. & Crossley, M. The regulation of human globin promoters by CCAAT box elements and the recruitment of NF-Y. Biochim. Biophys. Acta BBA-Gene Regul. Mech. 1860, 525-536 (2017).
12. Weber, L. et al. Editing a γ-globin repressor binding site restores fetal hemoglobin synthesis and corrects the sickle cell disease phenotype. Sci. Adv. 6, eaay9392 (2020).
13. Wang, L. et al. Reactivation of γ-globin expression through Cas9 or base editor to treat β-hemoglobinopathies. Cell Res. 30, 276-278 (2020).
14. Li, C. et al. In vivo HSPC gene therapy with base editors allows for efficient reactivation of fetal γ-globin in β-YAC mice. Blood Adv. 5, 1122-1135 (2021).
15. Mayuranathan, T. Adenosine Base Editing of γ-Globin Promoters Induces Fetal Hemoglobin and Inhibit Erythroid Sickling. in (ASH, 2020).
16. Milyavsky, M. et al. A Distinctive DNA Damage Response in Human Hematopoietic Stem Cells Reveals an Apoptosis-Independent Role for p53 in Self-Renewal. Cell Stem Cell 7, 186-197 (2010).
17. Cromer, M. K. et al. Global Transcriptional Response to CRISPR/Cas9-AAV6-Based Genome Editing in CD34+ Hematopoietic Stem and Progenitor Cells. Mol. Ther. J. Am. Soc. Gene Ther. 26, 2431-2442 (2018).
18. Schiroli, G. et al. Precise Gene Editing Preserves Hematopoietic Stem Cell Function following Transient p53-Mediated DNA Damage Response. Cell Stem Cell 24, 551-565.e8 (2019).
19. Haapaniemi, E., Botla, S., Persson, J., Schmierer, B. & Taipale, J. CRISPR-Cas9 genome editing induces a p53-mediated DNA damage response. Nat. Med. 24, 927-930 (2018).
20. Kosicki, M., Tomberg, K. & Bradley, A. Repair of double-strand breaks induced by CRISPR-Cas9 leads to large deletions and complex rearrangements. Nat. Biotechnol. 36, 765-771 (2018).
21. Boutin, J. et al. CRISPR-Cas9 globin editing can induce megabase-scale copy-neutral losses of heterozygosity in hematopoietic cells. Nat. Commun. 12, 4922 (2021).
22. Leibowitz, M. L. et al. Chromothripsis as an on-target consequence of CRISPR-Cas9 genome editing. Nat. Genet. 53, 895-905 (2021).
23. Turchiano, G. et al. Quantitative evaluation of chromosomal rearrangements in gene-edited human stem cells by CAST-Seq. Cell Stem Cell 28, 1136-1147.e5 (2021).
24. Koblan, L. W. et al. Improving cytidine and adenine base editors by expression optimization and ancestral reconstruction. Nat. Biotechnol. 36, 843-846 (2018).
25. Antoniou, P. et al. Base-editing-mediated dissection of a γ-globin cis-regulatory element for the therapeutic reactivation of fetal hemoglobin expression. Nat. Commun. 13, 6618 (2022).
26. Marktel, S. et al. Intrabone hematopoietic stem cell gene therapy for adult and pediatric patients affected by transfusion-dependent β-thalassemia. Nat. Med. 25, 234-241 (2019).
27. Magrin, E. et al. Long-term outcomes of lentiviral gene therapy for the β-hemoglobinopathies: the HGB-205 trial. Nat. Med. 28, 81-88 (2022).
28. Laszlo, G. S., Estey, E. H. & Walter, R. B. The past and future of CD33 as therapeutic target in acute myeloid leukemia. Blood Rev. 28, 143-153 (2014).
29. Laszlo, G. S. et al. Expression and functional characterization of CD33 transcript variants in human acute myeloid leukemia. Oncotarget 7, 43281-43294 (2016).
30. Kim, M. Y. et al. Genetic Inactivation of CD33 in Hematopoietic Stem Cells to Enable CAR T Cell Immunotherapy for Acute Myeloid Leukemia. Cell 173, 1439-1453.e19 (2018).
31. Knapp, D. J. H. F. et al. Single-cell analysis identifies a CD33+ subset of human cord blood cells with high regenerative potential. Nat. Cell Biol. 20, 710-720 (2018).
32. Richter, M. F. et al. Phage-assisted evolution of an adenine base editor with improved Cas domain compatibility and activity. Nat. Biotechnol. 38, 883-891 (2020).
33. Kluesner, M. G. et al. EditR: A Method to Quantify Base Editing from Sanger Sequencing. CRISPR J. 1, 239-250 (2018).
34. Brinkman, E. K., Chen, T., Amendola, M. & van Steensel, B. Easy quantitative assessment of genome editing by sequence trace decomposition. Nucleic Acids Res. 42, e168-e168 (2014).

1. A method for editing DNA in a eukaryotic cell comprising contacting said eukaryotic cell with a gene editing platform that comprises (a) at least one base-editing enzyme, and (b) a plurality of guide RNA molecules which guide the at least one base-editing enzyme to a plurality of target sequences, wherein at least one guide RNA molecule guides the at least one base-editing enzyme to a target sequence in a gene encoding CD33 so as to knock-down expression of CD33.

2. The method of claim 1 wherein the eukaryotic cell is selected from the group consisting of hematopoietic progenitor cells, hematopoietic stem cells (HSCs), pluripotent cells (PS) and induced pluripotent stem cells (iPS).

3. The method of claim 1 wherein the at least one base-editing enzyme is a cytosine base-editing enzyme (CBE) or an adenine base-editing enzyme (ABE).

4. The method according to claim 1, wherein the plurality of guide RNA molecules comprises i) at least one guide RNA molecule which guides the at least one base-editing enzyme to a target sequence of interest and ii) at least one guide RNA molecule which guides the at least one base-editing enzyme to a target sequence in the gene encoding for CD33 so as to knock-down the expression of CD33.

5. The method according to claim 1, wherein the plurality of guide RNA molecules comprises at least one guide RNA molecule which targets the CD33 gene and i) disrupts a start codon when coupled with a cytosine base editor (CBE) or ii) disrupts a splice donor site of exon1-2 when coupled either with the CBE or an adenine base editor (ABE) and/or iii) disrupts a splice acceptor site of exon1-2 when coupled with the ABE.

6. The method according to claim 1, wherein the at least one guide RNA molecule targets a nucleic acid sequence selected from Table 1.

7. The method according to claim 1, wherein the plurality of guide RNA molecules comprises at least one guide RNA molecule that targets CD33 and disrupts a splice donor site of exon1-2 when coupled either with a CBE or an ABE.

8. The method of claim 7 wherein the at least one guide RNA molecule targets the nucleic acid sequence as set forth in SEQ ID NO:13.

9. The method according to claim 1, wherein the gene editing platform comprises at least one adenine-base editor (ABE), and wherein the plurality of guide RNA molecules guide the at least one ABE to a plurality of target sequences, wherein at least one of the plurality of guide RNA molecules guides the ABE to the target sequence as set forth in SEQ ID NO:13.

10. A method for increasing fetal hemoglobin content in a eukaryotic cell, comprising editing the DNA of the eukaryotic cell by the method of claim 1.

11. The method of claim 10 wherein the gene editing platform edits an HBG1 or an HBG2 promoter and subsequently increases expression of γ-globin.

12. A method of repressing expression of α-globin in a eukaryotic cell, comprising editing the DNA of the eukaryotic cell by the method of claim 1.

13. A method of restoring normal expression of β-globin in a eukaryotic cell, comprising editing the DNA of the eukaryotic cell by the method of claim 1.

14. A population of edited eukaryotic cells obtained by the method according to claim 1.

15. A method of preparing a substantially pure population of edited eukaryotic cells comprising the steps of i) editing a population of eukaryotic cells by the method according to claim 1 and ii) enriching the population of edited eukaryotic cells that is negative for CD33.

16. A substantially pure population of edited eukaryotic cells obtained by the method of claim 15.

17. A method of therapy in a patient in need thereof, the method comprising transplanting a therapeutically effective amount of the population of edited eukaryotic cells of claim 14.

18. The method of claim 17 wherein the patient suffers from a β-hemoglobinopathy.

19. The method of claim 18 that further comprises administering to the patient a therapeutically effective amount of an agent that is capable of depleting CD33 positive cells.

20. The method of claim 19 wherein the agent is an antibody that binds to CD33 and depletes CD33+ cells.

21. The method of claim 20 wherein the antibody is conjugated to a cytotoxic moiety.

22. The method of claim 20 wherein the antibody is Gemtuzumab ozogamicin.

23. A kit comprising (a) at least one base-editing enzyme or a polynucleotide encoding the at least one base-editing enzyme and (b) a plurality of guide RNA molecules that guide the at least one base-editing enzyme to a plurality of target sequences, wherein at least one guide RNA molecule guides the base-editing enzyme(s) to a target sequence in a gene encoding CD33 so as to knock-down expression of CD33.

24. The kit of claim 23 wherein the at least one base-editing enzyme is an adenine-base editor (ABE) or a polynucleotide encoding the ABE, and wherein at least one guide RNA molecule is guides the ABE to the target sequence as set forth in SEQ ID NO:13.


SEQUENCE LISTING (21 sequences)

SEQ ID NO: 1 - protein, 364 aa, Homo sapiens
MPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 2 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 3 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 4 - protein, 167 aa, Staphylococcus aureus
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 5 - protein, 5 aa, synthetic construct
GGGGS

SEQ ID NO: 6 - protein, 5 aa, synthetic construct
EAAAK

SEQ ID NO: 7 - protein, 16 aa, synthetic construct
SGSETPGTSESATPES

SEQ ID NO: 8 - protein, 218 aa, synthetic construct
DIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 443 aa, synthetic construct
EVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - DNA, 20 bp, Homo sapiens
  source 1..20: mol_type = genomic DNA
gtggggaaggggcccccaag

SEQ ID NO: 11 - DNA, 20 bp, Homo sapiens
  source 1..20: mol_type = genomic DNA
gccccttccccacactatct

SEQ ID NO: 12 - DNA, 20 bp, Homo sapiens
  source 1..20: mol_type = genomic DNA
agcggcatgtctgaggaagc

SEQ ID NO: 13 - DNA, 20 bp, Homo sapiens
  source 1..20: mol_type = genomic DNA
cactcacctgcccacagcag

SEQ ID NO: 14 - DNA, 20 bp, Homo sapiens
  source 1..20: mol_type = genomic DNA
ccccacaggggccctggcta

SEQ ID NO: 15 - DNA, 30 bp, synthetic construct
  source 1..30: mol_type = other DNA
aaaaacggctgacaaaagaagtcctggtat

SEQ ID NO: 16 - DNA, 30 bp, synthetic construct
  source 1..30: mol_type = other DNA
ataacctcagacgttccagaagcgagtgtg

SEQ ID NO: 17 - DNA, 21 bp, synthetic construct
  source 1..21: mol_type = other DNA
caatctgtgtggaggggacaa

SEQ ID NO: 18 - DNA, 20 bp, synthetic construct
  source 1..20: mol_type = other DNA
aactggggagttcttgtcgt

SEQ ID NO: 19 - DNA, 20 bp, Homo sapiens
  source 1..20: mol_type = genomic DNA
gcttcctcagacatgccgct

SEQ ID NO: 20 - DNA, 20 bp, Homo sapiens
  source 1..20: mol_type = genomic DNA
ctgctgtgggcaggtgagtg

SEQ ID NO: 21 - DNA, 20 bp, Homo sapiens
  source 1..20: mol_type = genomic DNA
ggggtgtccccgggaccgat